(19) United States
(12) Patent Application Publication
(10) Pub. No.: US 2022/0387483 A1
(43) Pub. Date: Dec. 8, 2022

(54) USE OF IODIDE COMPOUNDS FOR THE TREATMENT AND PREVENTION OF CHEMOTHERAPY-ASSOCIATED CACHEXIA AND CARDIOTOXICITY

(71) Applicants: FARADAY PHARMACEUTICALS, INC., Seattle, WA (US); FRED HUTCHINSON CANCER CENTER, Seattle, WA (US)

(72) Inventors: Stephen A. HILL, Seattle, WA (US); Michael Andrew INSKO, Seattle, WA (US); Michael L. MORRISON, Seattle, WA (US); Akiko IWATA, Seattle, WA (US); Mark B. ROTH, Seattle, WA (US)

(21) Appl. No.: 17/774,099

(22) PCT Filed: Nov. 3, 2020
(86) PCT No.: PCT/US2020/058756

§ 371 Date: May 3, 2022

Related U.S. Application Data
(60) Provisional application No. 62/930,244, filed on Nov. 4, 2019.

(51) Int. Cl. A61K 33/18; A61K 31/704; A61K 33/243; A61K 31/675; A61K 39/395; A61K 31/337; A61K 31/7048; A61K 31/513; A61K 31/519; A61K 31/4745; A61P 21/00

(52) U.S. Cl. CPC A61K 33/18; A61K 31/704; A61K 33/243; A61K 31/675; A61K 39/3955; A61K 31/337; A61K 31/7048; A61K 31/513; A61K 31/519; A61K 31/4745; A61P 21/00

(57) ABSTRACT

The present invention relates to the use of iodide compounds, to treat and prevent cachexia and cardiotoxicity resulting from treatment with an anti-cancer therapy.

### CROSS-REFERENCE TO RELATED APPLICATIONS

This application claims priority to U.S. provisional application No. 62/930,244, filed Nov. 4, 2019, which is incorporated herein by reference in its entirety.

### GOVERNMENT INTEREST STATEMENT

This invention was made with government support under W911NF-14-1-0255 awarded by the Army Research Office. The government has certain rights in the invention.

### FIELD OF THE INVENTION

This disclosure relates to methods of using iodide, e.g., sodium iodide, for treating or preventing chemotherapy-associated cachexia and cardiotoxicity.

### BACKGROUND OF THE INVENTION

Cachexia is a complex syndrome associated with underlying illness causing ongoing muscle loss. A range of diseases can cause cachexia, most commonly cancer, congestive heart failure, chronic obstructive pulmonary disease, chronic kidney disease, chronic liver disease and AIDS. In addition, medical treatments, such as chemotherapeutic agents, can cause cachexia. Cachexia can be very serious and may complicate treatment for the condition that caused it and lower the response to treatment. For example, patients with cancer who have cachexia are less able to tolerate chemotherapy and other therapies. As a result of these complications, patients with cachexia have a lower quality of life and a worse clinical outlook.

There are three main categories of cachexia. Precachexia is defined as a loss of up to 5 percent of body weight while not trying to lose weight and having a known illness or disease. Cachexia is a loss of more than 5 percent of body weight over 12 months or less, while not trying to lose weight and having a known illness or disease. Several other criteria include loss of muscle strength, decreased appetite, fatigue, and inflammation. Refractory cachexia applies to individuals with cancer, and includes weight loss, muscle loss, loss of function, plus a failure to respond to cancer treatment. In some instances, cachexia is associated with a loss of cardiac muscle tissue, loss of skeletal muscle tissue, and/or loss of fat. These can result in loss of strength and reduced cardiac function. Thus, agents that cause cachexia can result in cardiotoxicity.

Like cachexia, cardiotoxicity is also a serious adverse effect of cancer treatment, including treatment with a variety of chemotherapeutic agents, such as anthracyclines, fluorouracil, taxanes, monoclonal antibodies, and tyrosine kinase inhibitors. Different types of cardiotoxicity include reversible (type 2), irreversible (type 1), acute, chronic, and late-onset. Cardiotoxicity may be defined into four categories: 1) directed cytotoxic effects of chemotherapy and associated cardiac dysfunction (associated with, e.g., alkylating agents, anthracyclines, interferon alpha, monoclonal antibodies, tyrosine kinase inhibitors); 2) cardiac ischemia (associated with, e.g., antitumor antibiotics, fluorouracil, topoisomerase inhibitors); 3) cardiac arrhythmias (associated with, e.g., anthracyclines, other agents); and 4) pericarditis (associated with, e.g., bleomycin, cyclophosphomide, cytarabine). Cardiotoxicity may result in cardiac dysfunction, which may be determined based on clinical symptoms or use of echocardiogram or electrocardiogram (EKG).

There is clearly an unmet need for specific treatments to treat or reverse cachexia and cardiotoxicity, including cachexia and cardiotoxicity associated with cancer treatments. The present disclosure addresses this need.

### BRIEF SUMMARY OF THE INVENTION

In certain embodiments, the disclosure provides a method for treating, reducing the severity of, or preventing cachexia or cardiotoxicity associated with or resulting from treatment of a subject with an anti-cancer therapy, comprising providing to the subject an effective amount of an iodide in combination with the anti-cancer therapy. In particular embodiments, the cachexia is one or more of precachexia, cachexia, or refractory cachexia. In some embodiments, the method is for treating, reducing the severity of, or preventing cardiotoxicity, and in some embodiments, the method is for treating, reducing the severity of, or preventing cachexia, optionally cachexia of skeletal muscle or cachexia of cardiac muscle. In particular embodiments, the subject is being treated for a cancer selected from the group consisting of: pancreatic cancer, bladder cancer, colorectal cancer, breast cancer, prostate cancer, renal cancer, hepatocellular cancer, lung cancer, ovarian cancer, cervical cancer, gastric cancer, esophageal cancer, head and neck cancer, melanoma, neuroendocrine cancer, central nervous system cancer, brain cancer, bone cancer, soft tissue sarcoma, non-small cell lung cancer, small-cell lung cancer, colon cancer, carcinoma, sarcoma, lymphoma, or leukemia. In various embodiments, the anti-cancer therapy comprises treatment with a chemotherapeutic agent. In particular embodiments, the chemotherapeutic agent is selected from the group consisting of: anthracyclines (optionally doxorubicin), cisplatin, cyclophosphamide, trastuzumab, paclitaxel, CPT-11, adriamycin, etoposide, 5-fluorouracil, and methotrexate. In one embodiment, the chemotherapeutic agent is an anthracycline, e.g., doxorubicin. In one embodiment, the chemotherapeutic agent is cisplatin. In certain embodiments, the anti-cancer therapy comprises radiation therapy. In particular embodiments of any of the methods, the iodide is sodium iodide. In some embodiments, the iodide, optionally sodium iodide, is provided to the subject in an amount sufficient to increase the blood concentration of the iodide in the subject by at least five-fold, at least ten-fold, at least 50-fold, at least 100-fold, at least 500-fold, at least 1000-fold, at least 10,000-fold, or at least 100,000-fold. In some embodiments, the iodide, optionally sodium iodide, and the anti-cancer agent are present in the subject during an overlapping time period. In some embodiments, the iodide, optionally sodium iodide, is provided to the subject before and/or during treatment of the subject with the anti-cancer agent. In particular embodiments, the subject is provided with less than or equal to about 10 mg/kg of the iodide, optionally about 1.0 mg/kg or about 2.0 mg/kg of the iodide. In some embodiments, the iodide, optionally sodium iodide, is provided to the subject at a dose of about 0.5 mg/kg to 5.0 mg/kg daily for a period of time during treatment of the subject with the anti-cancer agent. In some embodiments, the iodide, optionally sodium iodide, is provided to the subject as an intravenous bolus, optionally during a time period of about one hour to about one minute prior to treatment of the subject with the anticancer agent. In certain embodiments, the iodide is present in a stable liquid pharmaceutical composition comprising the iodide compound and a pharmaceutically acceptable carrier, diluent, or excipient. In some embodiments, at least 90% of the iodide in the composition is present in a reduced form for at least one hour, at least one week, at least one month, or at least six months when stored at room temperature. In further embodiments, the composition comprising the iodide comprises one or more of a reducing agent, a tonicity agent, a stabilizer, a surfactant, a lycoprotectant, a polyol, an antioxidant, or a preservative. In various embodiments, the iodide is provided to the subject orally or parenterally. In some embodiments, multiple doses of the iodide are provided to the subject.

In particular embodiments of any of the methods disclosed herein, the treatment with the iodide, optionally sodium iodide, results in a decreased loss or an increase in mean body weight as compared to in the absence of treatment with the iodide. In some embodiments, the treatment with the iodide, optionally sodium iodide, results in a decreased loss or an increase in tumor-free body weight as compared to in the absence of treatment with the iodide. In some embodiments, the treatment with the iodide, optionally sodium iodide, results in a decreased loss or an increase in liver weight, heart weight, and/or epididymal fat weight as compared to in the absence of treatment with the iodide. In some embodiments, the treatment with the iodide, optionally sodium iodide, results in a decreased loss or an increase in a muscle weight as compared to in the absence of treatment with the iodide. In particular embodiments, the muscle is tibialis anterior muscle. In some embodiments, the treatment with the iodide, optionally sodium iodide, results in decreased serum triglyceride levels, decreased serum VLDL levels, or increased serum LDL levels as compared to in the absence of treatment with the iodide. In some embodiments, the treatment with the iodide, optionally sodium iodide, results in a decreased tumor weight as compared to in the absence of treatment with the iodide.

In some embodiments of any of the methods disclosed herein, the iodide, e.g., NaI, or the composition is provided to the subject as a bolus dose prior to, concurrent with, or during an overlapping time period with chemotherapy, e.g., treatment with a chemotherapeutic agent, optionally wherein the bolus dose comprises less than or equal to about 10 mg/kg, optionally about 1.0 mg/kg or 2.0 mg/kg subject weight. In certain embodiments, the iodide, e.g., NaI, is provided to the subject as a bolus dose once a day for up to one day, two days, three days, four days, five days, six days, or seven days, or the duration of the chemotherapy treatment. In some embodiments, the iodide compound, e.g., NaI, or the composition is provided to the subject following one or more treatments, e.g. with a chemotherapeutic agent. In particular embodiments, the iodide compound is sodium iodide. In some embodiments, the subject is provided with the compound, e.g., NaI, via repeat daily doses of about 1 mg/kg or 2 mg/kg for several days, e.g., about 3 days, about 4 days, about 5 days, or about 1 week. In certain embodiments, the subject is provided with about 1000-fold the recommended daily allowance of NaI.

### BRIEF DESCRIPTION OF THE DRAWINGS

FIG. 1 shows the CT26 cachexia study design.

FIG. 2 shows mean tumor volume and tumor growth kinetics. Values are expressed as Mean±SEM of 10-13 animals in each group. Statistical analysis carried out by Two-way ANOVA followed by Bonferroni post tests using Graph Pad Prism (Version.5). *** p<0.001 when respective test groups (FDY-5301 & Bucindolol) were compared with vehicle control group. *On day 14, 1 h post dosing, blood sampling was carried out: 3 animals from group 2, 3 & 5. Plasma was separated and stored at −80° C.

FIG. 3 shows % mean body weight change. Values are expressed as Mean±SEM of 10-13 animals in each group. Based on cage side observations, there were no visible signs of abnormal behavior or clinical symptoms in any of the treated groups.

FIGS. 4A-4C show mean body weight (FIG. 4A), tumor-free body weight (FIG. 4B), and % tumor-free body weight change (FIG. 4C). Values are expressed as Mean±SEM of 10-13 animals in each group. Statistical analysis carried out by Two-way ANOVA followed by Bonferroni post tests using Graph Pad Prism (Version.5). *** p<0.001 when respective test groups (FDY-5301 & Bucindolol) were compared with vehicle control group.

FIG. 5 shows mean feed weight (g/mice/day). For each time point, the bars from left to right correspond to the legend from top to bottom.

FIGS. 6A-6B show mean tumor weight (FIG. 6A) and body weight—tumor weight (FIG. 6B). For FIG. 6A, values are expressed as Mean±SEM of 10 animals in each group Statistical analysis was carried out by one way ANOVA using Graph Pad Prism (Version.5). ** p<0.01 and *** p<0.001 when respective test groups (FDY-5301 & Bucindolol) were compared with vehicle control group. For FIG. 6B, values are expressed as Mean±SEM of 10 animals in each group Statistical analysis was carried out by unpaired t-test & one way ANOVA using Graph Pad Prism (Version.5), # indicates p<0.001 when normal was compared with vehicle control group; *** p<0.001 when respective test groups (FDY-5301 & Bucindolol) were compared with vehicle control group. ** p<0.01 and *** p<0.001 when respective test groups (FDY-5301 & Bucindolol) were compared with vehicle control group.

FIG. 7 shows mean organ weight for the indicated organs. Values are expressed as Mean±SEM of 10 animals in each group Statistical analysis was carried out by unpaired t-test and one way ANOVA using Graph Pad Prism (Version.5). For liver, # (** p<0.01) when normal was compared with vehicle control group, and *** p<0.001 when respective test groups (FDY-5301 & Bucindolol) were compared with vehicle control group. For heart, # (*** p<0.001) when normal was compared with vehicle control group, * p<0.05, and ** p<0.01 when respective test groups (FDY-5301 & Bucindolol) were compared with vehicle control group. For lung, # (* p<0.05) when normal was compared with vehicle control group, and ns (Non-significant) when respective test groups (FDY-5301 & Bucindolol) were compared with vehicle control group. For spleen, # (*** p<0.001) when normal was compared with vehicle control group, and ns (Non-significant) when respective test groups (FDY-5301 & Bucindolol) were compared with vehicle control group.

FIG. 8 shows mean muscle weight for the indicated muscles. Values are expressed as Mean±SEM of 10 animals in each group Statistical analysis was carried out by unpaired t-test and one way ANOVA using Graph Pad Prism (Version.5). For kidney, ns (Non-significant) when normal was compared with vehicle control group, and ns (Non-significant) when respective test groups (FDY-5301 & Bucindolol) were compared with vehicle control group. For epipididymal fat, # (*** p<0.001) when normal was compared with vehicle control group, * p<0.05 & ** p<0.01 when respective test groups (FDY-5301 & Bucindolol) were compared with vehicle control group.

FIGS. 9A-9K show biochemical analysis of serum levels of the indicated lipids and proteins. Values are expressed as Mean±SEM of 10 animals in each group Statistical analysis was carried out by unpaired t-test and one way ANOVA using Graph Pad Prism (Version.5). For FIG. 9A, # (** p<0.01) when normal was compared with vehicle control group, and ns (Non-significant) when respective test groups (FDY-5301 & Bucindolol) were compared with vehicle control group. For FIG. 9B, # (*** p<0.001) when normal was compared with vehicle control group, * p<0.05, and ** p<0.01 & *** p<0.001 when respective test groups (FDY-5301 & Bucindolol) were compared with vehicle control group. For FIG. 9C, # (* p<0.05) when normal was compared with vehicle control group, and ns (Non-significant) when respective test groups (FDY-5301 & Bucindolol) were compared with vehicle control group. For FIG. 9D, ns (Non-significant) when normal was compared with vehicle control group, and ns (Non-significant) when respective test groups (FDY-5301 & Bucindolol) were compared with vehicle control group. For FIG. 9E, # (*** p<0.001) when normal was compared with vehicle control group, and ns (Non-Significant) & *** p<0.001 when respective test groups (FDY-5301 & Bucindolol) were compared with vehicle control group. For FIG. 9F, # (*** p<0.001) when normal was compared with vehicle control group, and ns (Non-Significant) when respective test groups (FDY-5301 & Bucindolol) were compared with vehicle control group. For FIG. 9G, # (*** p<0.001) when normal was compared with vehicle control group, and ns (Non-Significant) & ** p<0.01 when respective test groups (FDY-5301 & Bucindolol) were compared with vehicle control group. For FIG. 9H, # (*** p<0.001) when normal was compared with vehicle control group, and ns (Non-Significant) & *** p<0.001 when respective test groups (FDY-5301 & Bucindolol) were compared with vehicle control group. For FIG. 9I, ns (Non-significant) when normal was compared with vehicle control group, and ns (Non-significant) when respective test groups (FDY-5301 & Bucindolol) were compared with vehicle control group. For FIG. 9J, ns (Non-significant) when normal was compared with vehicle control group, and ns (Non-significant) when respective test groups (FDY-5301 & Bucindolol) were compared with vehicle control group. For FIG. 9K, # (* p<0.05) when normal was compared with vehicle control group, and ns (Non-Significant) when respective test groups (FDY-5301 & Bucindolol) were compared with vehicle control group.

FIGS. 10A and 10B shows serum levels of TNF-α (FIG. 10A) and IL-6 (FIG. 10B). Values are expressed as Mean±SEM of 10 animals in each group. Statistical analysis was carried out by unpaired t-test and one way ANOVA using Graph Pad Prism (Version.5). For FIG. 10A, # (** p<0.01) when normal was compared with vehicle control group, and ns (Non-Significant) when respective test groups (FDY-5301 & Bucindolol) were compared with vehicle control group. For FIG. 10B, ns (Non-significant) when normal was compared with vehicle control group, and ns (Non-significant) when respective test groups (FDY-5301 & Bucindolol) were compared with vehicle control group.

FIG. 11 shows morphometric analysis of tibialis anterior. Values are expressed as Mean±SEM of 10 animals in each group. *** (p<0.001) when vehicle control was compared with normal control, ** (p<0.01) when FDY-ALZ-PUMP group was compared with vehicle control, and ns (Non-significant) when FDY-5301 and Bucindolol group were compared with vehicle control group.

FIG. 12 shows histopathological images of tibialis anterior from normal control group stained with Haematoxylin and Eosin and Periodic acid Schiff's under different magnifications, revealing normal muscle architecture.

FIG. 13 shows histopathological images of tibialis anterior from vehicle control group stained with Haematoxylin and Eosin and Periodic acid Schiff's under different magnifications, revealing reduced muscle fiber area when compared to the normal group.

FIG. 14 shows histopathological images of tibialis anterior from FDY-3501 (2 mg/kg) group stained with Haematoxylin and Eosin and Periodic acid Schiff's under different magnifications, revealing increased muscle fiber area when compared to the vehicle control group.

FIG. 15 shows histopathological images of tibialis anterior from Bucindolol (2 mg/kg) group stained with Haematoxylin and Eosin and Periodic acid Schiff's under different magnifications, revealing increased muscle fiber area when compared to the vehicle control group.

FIG. 16 shows histopathological images of tibialis anterior from FDY-3501 (40 ug/day; Alzet pump) group stained with Haematoxylin and Eosin and Periodic acid Schiff's under different magnifications, revealing increased muscle fiber area when compared to the vehicle control group.

FIG. 17 shows morphometric analysis of gastrocnemius. Values are expressed as Mean±SEM of 10 animals in each group. ns (Non-significant) when respective treatment groups were compared with vehicle control.

FIG. 18 shows histopathological images of gastrocnemius from normal control group stained with Haematoxylin and Eosin and Periodic acid Schiff's under different magnifications, revealing normal muscle architecture.

FIG. 19 shows histopathological images of gastrocnemius from vehicle control group stained with Haematoxylin and Eosin and Periodic acid Schiff's under different magnifications, revealing reduced muscle fiber area when compared to the normal group.

FIG. 20 shows histopathological images of gastrocnemius or from FDY-3501 (2 mg/kg) group stained with Haematoxylin and Eosin and Periodic acid Schiff's under different magnifications, revealing increased muscle fiber area when compared to the vehicle control group.

FIG. 21 shows histopathological images of gastrocnemius from Bucindolol (2 mg/kg) group stained with Haematoxylin and Eosin and Periodic acid Schiff's under different magnifications, revealing increased muscle fiber area when compared to the vehicle control group.

FIG. 22 shows histopathological images of gastrocnemius from FDY-3501 (40 ug/day; Alzet pump) group stained with Haematoxylin and Eosin and Periodic acid Schiff's under different magnifications, revealing increased muscle fiber area when compared to the vehicle control group.

FIG. 23 shows morphometric analysis of soleus. Values are expressed as Mean±SEM of 10 animals in each group. ns (Non-significant) when respective treatment groups were compared with vehicle control.

FIG. 24 shows histopathological images of soleus from normal control group stained with Haematoxylin and Eosin and Periodic acid Schiff's under different magnifications, revealing normal muscle architecture.

FIG. 25 shows histopathological images of soleus from vehicle control group stained with Haematoxylin and Eosin and Periodic acid Schiff's under different magnifications, revealing reduced muscle fiber area when compared to the normal group.

FIG. 26 shows histopathological images of soleus or from FDY-3501 (2 mg/kg) group stained with Haematoxylin and Eosin and Periodic acid Schiff's under different magnifications, revealing increased muscle fiber area when compared to the vehicle control group.

FIG. 27 shows histopathological images of soleus from Bucindolol (2 mg/kg) group stained with Haematoxylin and Eosin and Periodic acid Schiff's under different magnifications, revealing increased muscle fiber area when compared to the vehicle control group.

FIG. 28 shows histopathological images of soleus from FDY-3501 (40 ug/day; Alzet pump) group stained with Haematoxylin and Eosin and Periodic acid Schiff's under different magnifications, revealing increased muscle fiber area when compared to the vehicle control group.

FIG. 29 shows change in ejection fraction on days 7, 14, and 28, following FDY-5301 administered as a single i.v. bolus on day 0. At each time point, placebo is shown on the left, and FDY-5301 is shown on the right.

FIG. 30 shows change in ejection fraction on days 3, 7, and 14 following FDY-5301 administration as an i.v. bolus+continuous administration starting on day 0. At each time point, placebo is shown on the left, and FDY-5301 is shown on the right.

FIGS. 31A-B shows combined results of change in ejection fraction on days 3, 7, 14, and 28 following FDY-5301 administered as a single i.v. bolus and following FDY-5301 administration as an i.v. bolus+continuous administration starting on day 0, as a dot plot (FIG. 31A) and a line graph (FIG. 31B). In FIG. 31A, at each time point, placebo is shown on the left, and FDY-5301 is shown on the right.

### DETAILED DESCRIPTION OF THE INVENTION

The disclosure provides methods for treating, inhibiting, or reducing the severity of cachexia or cardiotoxicity in a subject in need thereof. As shown in the accompanying Examples, treatment of cancer patients with iodide resulted in reduced cachexia, including a reduced loss of body weight, a reduced loss of tumor-free body weight, and a reduced loss of liver, heart, and muscle weight. These results demonstrate the successful use of iodide to inhibit or reduce the severity of cachexia and cardiotoxicity, including but not limited to that associated with or resulting from cancer or cancer therapies.

Definitions and Abbreviations

Unless otherwise defined herein, scientific and technical terms used in this application shall have the meanings that are commonly understood by those of ordinary skill in the art. Generally, nomenclature used in connection with, and techniques of, chemistry, molecular biology, cell and cancer biology, immunology, microbiology, pharmacology, and protein and nucleic acid chemistry, described herein, are those well-known and commonly used in the art.

As used herein, the following terms have the meanings ascribed to them unless specified otherwise.

The term “including” is used to mean “including but not limited to.” “Including” and “including but not limited to” are used interchangeably.

The words “a” and “an” denote one or more, unless specifically noted.

By “about” is meant a quantity, level, value, number, frequency, percentage, dimension, size, amount, weight or length that varies by as much as 30, 25, 20, 15, 10, 9, 8, 7, 6, 5, 4, 3, 2 or 1% to a reference quantity, level, value, number, frequency, percentage, dimension, size, amount, weight or length. In any embodiment discussed in the context of a numerical value used in conjunction with the term “about,” it is specifically contemplated that the term about can be omitted.

Unless the context requires otherwise, throughout the present specification and claims, the word “comprise” and variations thereof, such as, “comprises” and “comprising” are to be construed in an open, inclusive sense, that is as “including, but not limited to”.

By “consisting of” is meant including, and limited to, whatever follows the phrase “consisting of.” Thus, the phrase “consisting of” indicates that the listed elements are required or mandatory, and that no other elements may be present.

By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements.

Reference throughout this specification to “one embodiment” or “an embodiment” means that a particular feature, structure or characteristic described in connection with the embodiment is included in at least one embodiment of the present invention. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

An “increased” or “enhanced” amount is typically a “statistically significant” amount, and may include an increase that is 1.1, 1.2, 1.3, 1.4, 1.5, 1.6, 1.7, 1.8, 1.9, 2, 2.5, 3, 3.5, 4, 4.5, 5, 6, 7, 8, 9, 10, 15, 20, 30, 40, or 50 or more times (e.g., 100, 500, 1000 times) (including all integers and decimal points in between and above 1, e.g., 2.1, 2.2, 2.3, 2.4, etc.) greater than an amount or level described herein.

A “decreased” or “reduced” or “lesser” amount is typically a “statistically significant” amount, and may include a decrease that is about 1.1, 1.2, 1.3, 1.4, 1.5, 1.6 1.7, 1.8, 1.9, 2, 2.5, 3, 3.5, 4, 4.5, 5, 6, 7, 8, 9, 10, 15, 20, 30, 40, or 50 or more times (e.g., 100, 500, 1000 times) (including all integers and decimal points in between and above 1, e.g., 1.5, 1.6, 1.7, 1.8, etc.) less than an amount or level described herein, for example an amount that is 10%, 20%, 30%, 40%, 50%, 60%, 70%, 80%, or 90% of an amount or level described herein.

A “composition” can comprise an active agent, e.g., sodium iodide, and a carrier, inert or active, e.g., a pharmaceutically acceptable carrier, diluent or excipient. A composition may be a pharmaceutical composition. In particular embodiments, the compositions are sterile, substantially free of endotoxins or non-toxic to recipients at the dosage or concentration employed.

“Pharmaceutical composition” refers to a formulation of a compound and a medium generally accepted in the art for the delivery of the biologically active compound to mammals, e.g., humans. Such a medium may include any pharmaceutically acceptable carriers, diluents or excipients therefore.

“Pharmaceutically acceptable carrier, diluent or excipient” includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent or emulsifier which has been approved by the United States Food and Drug Administration as being acceptable for use in humans or domestic animals.

The terms “mammal” and “subject” includes human and non-human mammals, such as, e.g., a human, mouse, rat, rabbit, monkey, cow, hog, sheep, horse, dog, and cat.

“Optional” or “optionally” means that the subsequently described event or circumstances may or may not occur, and that the description includes instances where said event or circumstance occurs and instances in which it does not.

“Iodide” and “a reduced form of iodine” both refer to iodide, which has a −1 valence state (e.g., NO).

“Therapeutically effective amount” refers to that amount of a compound or composition of the invention that, when administered to a subject, is sufficient to effect treatment, as defined below, of a disease, injury, or condition in the biological material, e.g., mammal, preferably a human. The amount of a compound or composition of the invention which constitutes a “therapeutically effective amount” may vary depending on the compound or composition, the disease, injury or condition and its severity, the manner of administration, and the age of the subject to be treated, but can be determined routinely by one of ordinary skill in the art having regard to his own knowledge and to this disclosure.

“Treating” or “treatment” as used herein covers the treatment of the disease, injury, or condition of interest, e.g., cachexia, in a biological material, e.g., mammal, preferably a human, having the disease or condition of interest, and includes: (i) preventing or inhibiting the disease, injury, or condition from occurring in a biological material, e.g., mammal, in particular, when such mammal is predisposed to the condition but has not yet been diagnosed as having it; (ii) reducing the severity or duration of the disease, injury or condition, e.g., when it occurs, e.g., in a mammal predisposed to the condition; (iii) inhibiting the disease, injury, or condition, i.e., arresting its development; (iv) relieving the disease, injury, or condition, i.e., causing regression of the disease or condition; or (v) relieving the symptoms resulting from the disease, injury, or condition. In certain embodiments, as used herein, the term “prevention” includes inhibiting or impeding the onset or progression of a disease or injury, or reducing the amount of injury or damage caused by a disease or injury. As used herein, the terms “disease,” “disorder,” and “condition” may be used interchangeably.

The term “anticancer agent” or “chemotherapeutic agent” is any drug that is effective in the treatment of a malignant, or cancerous disease. Effectiveness may mean inhibition, partial, or full remission, prolongation of life, improvement in quality of life, or cure.

The term “anticancer therapy” means any currently known therapeutic method for the treatment of cancer.

Methods of Treatment

The present disclosure includes methods and compositions related to the use of an iodide, e.g., I— or NaI, to treat, inhibit, or reduce the severity of cachexia or cardiotoxicity, e.g., cachexia or cardiotoxicity associated with a disease or a disease treatment. In particular embodiments, the cachexia or cardiotoxicity is associated with or results from cancer or a cancer treatment, such as treatment with a chemotherapeutic agent or radiation therapy. In certain embodiments, the cachexia is precachexia with weight loss up to 5% over 12 months and having a known illness or disease, e.g., cancer; cachexia with weight loss of 5% or greater over 12 month and having a known illness or disease, e.g., cancer; or refractory cachexia. In certain embodiments, the cardiotoxicity is reversible (type 2), irreversible (type 1), acute, chronic, or late-onset cardiotoxicity.

In one embodiment, the disclosure provides a method of treating, inhibiting, or reducing the severity of cachexia or cardiotoxicity in a subject being treated for a disease or disorder, comprising providing to the subject an effective amount of iodide, e.g., NaI. In particular embodiments, the disease or disorder is a tumor or cancer, and the subject is being treated with a cancer therapy, e.g., radiation therapy or chemotherapy. In particular embodiments, the cancer therapy, e.g., radiation therapy or chemotherapeutic agent, is associated with or can result in cachexia or cardiotoxicity. In particular examples, the cancer therapy is treatment with an anthracycline antibiotic, such as doxorubicin, or cisplatin, and/or the cancer being treated is a bladder, breast, lung, stomach, prostate, ovarian cancer, lymphoma (e.g., Hodgkin's lymphoma (Hodgkin's disease) or non-Hodgkin's lymphoma (cancer that begins in the cells of the immune system)), or a leukemia (cancer of the white blood cells). In certain embodiments, the cancer therapy is treatment with an anthracycline antibiotic, e.g., doxorubicin, and the cancer being treated is a leukemia, lymphoma, breast cancer, prostate cancer, ovarian cancer, or lung cancer, and treatment with iodide treats, inhibits, or reduces the severity of cachexia. In certain embodiments, the cancer therapy is treatment with an anthracycline antibiotic, e.g., doxorubicin, alone or in combination with cyclophosphamide, trastuzumab and/or paclitaxel, and the cancer being treated is a breast cancer, sarcoma, lymphoma, or leukemias, and treatment with iodide treats, inhibits, or reduces the severity of cardiotoxicity. In certain embodiments, the cancer therapy is treatment with an alkylating agent, e.g., cyclophosphamide, and the cancer being treated is a lymphoma, leukemia, or myeloma (e.g., multiple myeloma), and treatment with iodide treats, inhibits, or reduces the severity of cardiotoxicity. In certain embodiments, the cancer therapy is treatment with an inhibitor of microtubule polymerization, e.g., paclitaxel, and the cancer being treated is a breast cancer or a lung cancer, and treatment with iodide treats, inhibits, or reduces the severity of cardiotoxicity. In certain embodiments, the cancer therapy is treatment with a monoclonal antibody, e.g., trastuzumab, and the cancer being treated is a breast cancer or a gastric cancer, and treatment with iodide treats, inhibits, or reduces the severity of cardiotoxicity. In some embodiments, the subject is provided with the iodide compound, e.g., NaI, via repeat daily doses of about 1 mg/kg or 2 mg/kg for several days, e.g., about 3 days, about 4 days, about 5 days, or about 1 week, or for the duration of treatment with the cancer therapy.

In certain embodiments, the subject is provided with the iodide before the cancer therapy and/or during an overlapping time period with the cancer therapy. For example, the subject may be provided with a bolus dose of NaI before undergoing radiation therapy or a chemotherapeutic treatment, such as an intravenous infusion. In addition, or instead, the subject may be provided with NaI over the course of the treatment. In particular embodiments, the subject is provided with 0.5 mg/kg-5 mg/kg (e.g., 1 mg/kg or 2 mg/kg) of iodide, e.g., NaI, daily for the duration of the cancer therapy. In particular embodiments, the iodide, e.g., sodium iodide is provided to the subject as a bolus or via an osmotic pump, e.g., as disclosed in the accompanying examples. In certain embodiments, the subject is provided with an intravenous bolus between one hour up to one minute prior to administration of the cancer therapy.

In particular embodiments, treatment with the NaI results in reduced cachexia resulting from the cancer therapy, which may be demonstrated in a variety of ways, such as, e.g., reduced total weight loss, reduced liver weight loss, reduced heart weight loss, or reduced muscle weight loss. In particular embodiments, reduced cachexia is evidenced as a total weight loss of less than 10% or less than 5% as compared to the subject's baseline total weight before treatment with the cancer therapy. In certain embodiments, the cancer therapy is treatment with an anthracycline antibiotic, e.g., doxorubicin, and the cancer being treated is a leukemia, lymphoma, breast cancer, prostate cancer, ovarian cancer, or lung cancer. In some embodiments, treatment with NaI reduces loss of skeletal muscle and/or cardiac muscle following the cancer therapy, e.g., chemotherapy or radiation therapy. In particular examples, the cancer therapy is treatment with an anthracycline antibiotic, such as doxorubicin, or cisplatin. In particular embodiments, the iodide, e.g., sodium iodide is provided to the subject as a bolus or via an osmotic pump, e.g., as disclosed in the accompanying examples. In particular examples, the cancer therapy is treatment with an anthracycline antibiotic, such as doxorubicin, or cisplatin.

In particular embodiments, treatment with the NaI results in reduced cardiotoxicity resulting from the cancer therapy, which may be demonstrated in a variety of ways, such as by less of a decline or no decline in systolic function as quantified through measurement of left ventricular ejection fraction (LVEF), e.g., a reduced or lessened LVEF) reduction. In particular embodiments, treatment with the NaI results in the treated subject's LVEF either not decreasing or decreasing less than 10 percentage points from baseline. In certain embodiments, a LVEF) reduction of less than 10% or less than 5% as compared to a normal range or the subject's baseline prior to treatment with the cancer therapy is an indication of reduced cardiotoxicity. In certain embodiments, reduced cardiotoxicity may be demonstrated via echocardiographic measurement of global longitudinal strain (GLS). Global systolic longitudinal myocardial strain (GLS) on echocardiography has emerged as a reproducible indicator of early anthracycline-related myocardial dysfunction and future reduction in LVEF. A fall in GLS of 15% compared with baseline measurement is considered pathological and an early injury marker. In particular embodiments, treatment with the NaI results in the treated subject's GLS either not decreasing or decreasing less than 15%, e.g., less than 10% or less than 5% from baseline. In certain embodiments, a GSL reduction of less than 15% or 10% or less than 5% as compared to a normal range or the subject's baseline prior to treatment with the cancer therapy is an indication of reduced cardiotoxicity. Reduced cardiotoxicity may also be measured as less cardiac dysfunction, which may be determined based on clinical symptoms or the use of echocardiogram or electrocardiogram (EKG). In particular examples, the cardiotoxicity is type 1, and the cancer therapy is treatment with an anthracycline antibiotic (such as doxorubicin, daunorubicin, epirubicin, or idarubicin), an alkylating agent (such as busulfan, carboplatin, carmustine, chlormethine, cisplatin, cyclophosphamide, or mitomycin), a taxane (such as docetaxel, cabazitaxel, paclitaxel), a topoisomerase inhibitor (such as etoposide, tretinoin, or vinca alkaloids), or anantimetabolite (such as cladribine, cyarabine, or 5-FU). In certain embodiments, the cancer therapy is treatment with an anthracycline antibiotic, e.g., doxorubicin, and the cancer being treated is a leukemia, lymphoma, breast cancer, prostate cancer, ovarian cancer, or lung cancer. In certain embodiments, the cancer therapy is treatment with an anthracycline antibiotic, e.g., doxorubicin, alone or in combination with cyclophosphamide, trastuzumab and/or paclitaxel), and the cancer being treated is a breast cancer, sarcoma, lymphoma, or leukemias. In certain embodiments, the cancer therapy is treatment with an alkylating agent, e.g., cyclophosphamide, and the cancer being treated is a lymphoma, leukemia, or myeloma (e.g., multiple myeloma). In certain embodiments, the cancer therapy is treatment with an inhibitor of microtubule polymerization, e.g., paclitaxel, and the cancer being treated is a breast cancer or a lung cancer. In certain embodiments, the cancer therapy is treatment with a monoclonal antibody, e.g., trastuzumab, and the cancer being treated is a breast cancer or a gastric cancer.

In particular embodiments, the iodide, e.g., sodium iodide is provided to the subject as a bolus or via an osmotic pump, e.g., as disclosed in the accompanying examples.

In one embodiment, the disclosure provides a method of treating a disease or disorder in a subject in need thereof, comprising providing to the subject an effective amount of iodide, e.g., NaI, in combination with a therapy for the disease or disorder, wherein the iodide is effective in treating, inhibiting, or reducing the severity of cachexia or cardiotoxicity in the subject being treated. In particular embodiments, the disease or disorder is a tumor or cancer, and the therapy is a cancer therapy, e.g., radiation therapy or chemotherapy. In particular embodiments, the cancer therapy, e.g., radiation therapy or chemotherapeutic agent, is associated with or can result in cachexia or cardiotoxicity. In particular examples, the cancer therapy is treatment with an anthracycline antibiotic, such as doxorubicin, or cisplatin, and/or the cancer being treated is a bladder, breast, lung, stomach, prostate, ovarian cancer, lymphoma (e.g., Hodgkin's lymphoma (Hodgkin's disease) or non-Hodgkin's lymphoma (cancer that begins in the cells of the immune system)), or a leukemia (cancer of the white blood cells). In certain embodiments, the cancer therapy is treatment with an anthracycline antibiotic, e.g., doxorubicin, and the cancer being treated is a leukemia, lymphoma, breast cancer, prostate cancer, ovarian cancer, or lung cancer, and treatment with iodide treats, inhibits, or reduces the severity of cachexia. In certain embodiments, the subject is provided with the iodide before the cancer therapy and/or during an overlapping time period with the cancer therapy. For example, the subject may be provided with a bolus dose of NaI before undergoing radiation therapy or a chemotherapeutic treatment, such as an intravenous infusion. In addition, or instead, the subject may be provided with NaI over the course of the treatment. In particular embodiments, the subject is provide with 0.5 mg/kg-5 mg/kg (e.g., 1 mg/kg or 2 mg/kg) of iodide, e.g., NaI, daily for the duration of the cancer therapy. In certain embodiments, the subject is provided with an intravenous bolus between one hour up to one minute prior to administration of the cancer therapy.

In particular embodiments, treatment with the NaI in combination with the cancer therapy results in reduced cachexia resulting from the cancer therapy, which may be demonstrated in a variety of ways, including but not limited to any described herein, such as, e.g., reduced total weight loss, reduced liver weight loss, reduced heart weight loss, or reduced muscle weight loss. In certain embodiments, the cancer therapy is treatment with an anthracycline antibiotic, e.g., doxorubicin, and the cancer being treated is a leukemia, lymphoma, breast cancer, prostate cancer, ovarian cancer, or lung cancer. In some embodiments, treatment with NaI reduces loss of skeletal muscle and/or cardiac muscle following the cancer therapy, e.g., chemotherapy or radiation therapy. In particular examples, the subject is provided with the iodide, e.g., NaI, in combination with an anthracycline antibiotic, such as doxorubicin, or cisplatin. The iodide and the chemotherapeutic agent may be provided in the same or separate compositions, at the same time or different times. In particular embodiments, the subject is provided with the iodide, e.g., NaI, and the chemotherapeutic agent during an overlapping period of time. In particular embodiments, the iodide, e.g., sodium iodide is provided to the subject as a bolus or via an osmotic pump, e.g., as disclosed in the accompanying examples. In particular embodiments, the subject is provided with 0.5 mg/kg-5 mg/kg (e.g., 1 mg/kg or 2 mg/kg) of iodide, e.g., NaI, daily for about or up to one day, two days, three days, four days, five days, six days, or seven days, or for the duration of the cancer therapy. In certain embodiments, the subject is provided with an intravenous bolus between one hour up to one minute prior to administration of the cancer therapy.

In particular embodiments, treatment with the NaI in combination with the cancer therapy results in reduced cardiotoxicity resulting from the cancer therapy, which may be demonstrated in a variety of ways, including but not limited to any described herein, such as, e.g., a reduced or lessened ejection-fraction (e.g., LVEF) reduction, e.g., an ejection-fraction (e.g., LVEF) reduction of <10% or less than 5%. Reduced cardiotoxicity may also be measured as less cardiac dysfunction, which may be determined based on clinical symptoms or the use of echocardiogram or electrocardiogram (EKG). In particular examples, the cardiotoxicity is type 1, and the cancer therapy is treatment with an anthracycline antibiotic (such as doxorubicin, daunorubicin, epirubicin, or idarubicin), an alkylating agent (such as busulfan, carboplatin, carmustine, chlormethine, cisplatin, cyclophosphamide, or mitomycin), a taxane (such as docetaxel, cabazitaxel, paclitaxel), a topoisomerase inhibitor (such as etoposide, tretinoin, or vinca alkaloids), or anantimetabolite (such as cladribine, cyarabine, or 5-FU). In certain embodiments, the cardiotoxicity is type 1, the cancer therapy is an anthracycline (e.g., doxorubicin, daunorubicin, epirubicin, or idarubicin), and the cancer is breast cancer, a gynecologic cancer, a sarcoma, or a lymphoma. In certain embodiments, the cardiotoxicity is type 2, and the cancer therapy is treatment with a monoclonal antibody, such as, e.g., trastuzumab, levacizumab, lapatinib, or sunitinib. In certain embodiments, the cancer therapy is treatment with an anthracycline antibiotic, e.g., doxorubicin, and the cancer being treated is a leukemia, lymphoma, breast cancer, prostate cancer, ovarian cancer, or lung cancer. In certain embodiments, the cancer therapy is treatment with an anthracycline antibiotic, e.g., doxorubicin, alone or in combination with cyclophosphamide, trastuzumab and/or paclitaxel), and the cancer being treated is a breast cancer, sarcoma, lymphoma, or leukemias. In certain embodiments, the cancer therapy is treatment with an alkylating agent, e.g., cyclophosphamide, and the cancer being treated is a lymphoma, leukemia, or myeloma (e.g., multiple myeloma). In certain embodiments, the cancer therapy is treatment with an inhibitor of microtubule polymerization, e.g., paclitaxel, and the cancer being treated is a breast cancer or a lung cancer. In certain embodiments, the cancer therapy is treatment with a monoclonal antibody, e.g., trastuzumab, and the cancer being treated is a breast cancer or a gastric cancer. In particular embodiments, the iodide, e.g., sodium iodide is provided to the subject as a bolus or via an osmotic pump, e.g., as disclosed in the accompanying examples.

In particular embodiments, the any of the disclosed methods comprising administering NaI to prevent or reduce cachexia and/or cardiotoxicity are practiced on a subject being treated with an anthracycline (e.g., doxorubicin, daunorubicin, epirubicin, idarubicin) for breast, ovarian, bladder, lung, gynecologic, sarcoma, lymphoma, leukemia, or gastric cancer or tumor. In one embodiment, the method is used to prevent or reduce cardiotoxicity.

In particular embodiments, the any of the disclosed methods comprising administering NaI to prevent or reduce cachexia and/or cardiotoxicity are practiced on a subject being treated with Pertuzumab for breast cancer. In one embodiment, the method is used to prevent or reduce cardiotoxicity.

In particular embodiments, the any of the disclosed methods comprising administering NaI to prevent or reduce cachexia and/or cardiotoxicity are practiced on a subject being treated with Trastuzumab or a derivative thereof for breast cancer. In one embodiment, the method is used to prevent or reduce cardiotoxicity.

In particular embodiments, the any of the disclosed methods comprising administering NaI to prevent or reduce cachexia and/or cardiotoxicity are practiced on a subject being treated with Bevacizumab for colorectal, lung, or glioblastoma cancer or tumor. In one embodiment, the method is used to prevent or reduce cardiotoxicity.

In particular embodiments, the any of the disclosed methods comprising administering NaI to prevent or reduce cachexia and/or cardiotoxicity are practiced on a subject being treated with Lapatinib for breast cancer. In one embodiment, the method is used to prevent or reduce cardiotoxicity.

In particular embodiments, the any of the disclosed methods comprising administering NaI to prevent or reduce cachexia and/or cardiotoxicity are practiced on a subject being treated with Sunitinib for gastrointestinal stromal tumor (GIST), renal, or pancreatic neuroendocrine tumor (NET) cancer or tumor. In one embodiment, the method is used to prevent or reduce cardiotoxicity.

In particular embodiments, the any of the disclosed methods comprising administering NaI to prevent or reduce cachexia and/or cardiotoxicity are practiced on a subject being treated with 5-fluorouracil (5FU) for breast, head and neck, anal, gastric, colon, or skin cancer or tumor. In one embodiment, the method is used to prevent or reduce cardiotoxicity. In one embodiment, the method is used to prevent or reduce cachexia.

In particular embodiments, the any of the disclosed methods comprising administering NaI to prevent or reduce cachexia and/or cardiotoxicity are practiced on a subject being treated with Capecitabine for breast, colon, or rectal cancer or tumor. In one embodiment, the method is used to prevent or reduce cardiotoxicity.

In particular embodiments, the any of the disclosed methods comprising administering NaI to prevent or reduce cachexia and/or cardiotoxicity are practiced on a subject being treated with Paclitaxel for ovarian, breast, lung, Kaposi, cervical, pancreas, or prostate cancer or tumor. In one embodiment, the method is used to prevent or reduce cardiotoxicity.

In particular embodiments, the any of the disclosed methods comprising administering NaI to prevent or reduce cachexia and/or cardiotoxicity are practiced on a subject being treated with Docetaxel for breast, head and neck, gastric, prostate, lung, or non-small cell lung cancer (NSCLC) cancer or tumor. In one embodiment, the method is used to prevent or reduce cardiotoxicity.

In particular embodiments, the any of the disclosed methods comprising administering NaI to prevent or reduce cachexia and/or cardiotoxicity are practiced on a subject being treated with Imatinib for Chronic myeloid leukemia (CML), Acute lymphocytic leukemia (ALL), myelodysplastic/myeloproliferative diseases or neoplasms (MDS/MPD), or GIST cancer or tumor. In one embodiment, the method is used to prevent or reduce cardiotoxicity.

In particular embodiments, the any of the disclosed methods comprising administering NaI to prevent or reduce cachexia and/or cardiotoxicity are practiced on a subject being treated with Cyclophosphamide for sarcoma, neuroblastoma, ovarian, breast, lung SCLC, lymphoma, multiple myeloma, or leukemia cancer or tumor. In one embodiment, the method is used to prevent or reduce cachexia.

In particular embodiments, the any of the disclosed methods comprising administering NaI to prevent or reduce cachexia and/or cardiotoxicity are practiced on a subject being treated with Cisplatin for breast, cervical, gut, ovarian, breast, or bladder cancer or tumor. In one embodiment, the method is used to prevent or reduce cachexia.

In particular embodiments, the any of the disclosed methods comprising administering NaI to prevent or reduce cachexia and/or cardiotoxicity are practiced on a subject being treated with Methotrexate for leukemia, breast, skin, head and neck, lung, or uterus cancer or tumor. In one embodiment, the method is used to prevent or reduce cachexia.

In particular embodiments, the any of the disclosed methods comprising administering NaI to prevent or reduce cachexia and/or cardiotoxicity are practiced on a subject being treated with Adriamycin for breast, ovarian, bladder, lung, gynecologic, sarcoma, lymphoma, leukemia, or gastric cancer or tumor. In one embodiment, the method is used to prevent or reduce cachexia.

In particular embodiments, the any of the disclosed methods comprising administering NaI to prevent or reduce cachexia and/or cardiotoxicity are practiced on a subject being treated with Etoposide for testicular, lung, lymphoma, leukemia, neuroblastoma, or ovarian cancer or tumor. In one embodiment, the method is used to prevent or reduce cachexia.

In particular embodiments, the any of the disclosed methods comprising administering NaI to prevent or reduce cachexia and/or cardiotoxicity are practiced on a subject being treated with Folfox for colorectal or gastric cancer or tumor. In one embodiment, the method is used to prevent or reduce cachexia.

In particular embodiments, the any of the disclosed methods comprising administering NaI to prevent or reduce cachexia and/or cardiotoxicity are practiced on a subject being treated with Folfox for colorectal cancer or tumor. In one embodiment, the method is used to prevent or reduce cachexia. In particular embodiments, the any of the disclosed methods comprising administering NaI to prevent or reduce cachexia are practiced on a subject being treated with an agent disclosed in Pin, F. et al., Cachexia induced by cancer and chemotherapy yield distinct perturbations to energy metabolism, Journal of Cachexia, Sarcopenia and Muscle 2019; 10: 140-154, which is incorporated by reference herein in its entirety.

In particular embodiments, the any of the disclosed methods comprising administering NaI to prevent or reduce cardiotoxicity are practiced on a subject being treated with an agent disclosed in Thomas, S. A., Chemotherapy Agents That Cause Cardiotoxicity, US Pharm. 2017; 42(9):HS24-HS33, which is incorporated by reference herein in its entirety.

In one embodiment, the disclosure provides a method of treating a tumor or cancer in a subject in need thereof, comprising providing to the subject an effective amount of iodide, e.g., NaI, wherein the iodide is effective in treating the cancer and/or reducing the size of a tumor or tumor volume. For example, tumor volume may be reduced by at least 5%, at least 10%, at least 20%, at least 30%, at least 40%, or at least 50%, as compared to tumor volume in the absence of treatment with the iodide. In particular embodiments, the iodide, e.g., sodium iodide is provided to the subject as a bolus or via an osmotic pump, e.g., as disclosed in the accompanying examples. In particular embodiments, the subject is provided with 0.5 mg/kg-5 mg/kg (e.g., 1 mg/kg or 2 mg/kg) of iodide, e.g., NaI, daily.

In particular embodiments, the methods are practiced on a mammalian subject at risk of or suffering cachexia or cardiotoxicity. In some embodiments, the mammalian subject may be selected from human beings, non-human primates, dogs, cats, horses, cattle, sheep, goats, and pigs. Human subjects might be male, female, adults, children, or seniors (65 and older). The mammalian subject can be one diagnosed with cancer, HIV infection, tuberculosis, chronic obstructive pulmonary disease, chronic heart failure, chronic renal failure, a hormone imbalance, severe trauma (e.g., burns), hypermetabolism (e.g., sustained elevated heart rate of at least 6 bpm over normal for a given subject), excessive sympathetic nerve activity, a hyper-inflammatory state (e.g., elevated CRP levels, increased IL-6 levels, increased TNF-alpha levels, and/or increased IFN-gamma levels), a >5 lb weight loss in the preceding two months, and/or an estimated daily caloric intake of <20 cal/kg.

In various embodiments of the methods disclosed herein, the subject being treated has cachexia resulting from a disease or disorder, including but not limited to, cancer (including but not limited to any type of cancer disclosed herein), congestive heart failure, chronic obstructive pulmonary disease, chronic kidney disease, chronic liver disease and AIDS.

In various embodiments of the methods disclosed herein, the subject being treated has cachexia or cardiotoxicity resulting from treatment for a disease or disorder, including but not limited to any of those disclosed herein. In particular embodiments, the disease or disorder is a tumor or a cancer, such as a metastatic cancer; solid tumor cancers; and stage II, III, or IV cancers, and including but not limited to any of those disclosed herein. Thus, in certain embodiments, the subject has cachexia or cardiotoxicity associated with or resulting from a cancer treatment, including but not limited to, treatment with a chemotherapeutic agent and/or treatment with radiation. In particular embodiments, the chemotherapeutic agent includes but is not limited to any of those disclosed herein.

Methods disclosed herein may be used to treat, inhibit, or reduce the severity of any sign or symptom of cachexia. Examples of such signs and symptoms include weakness, fatigue, gastrointestinal distress, sleep/wake disturbances, pain, listlessness, shortness of breath, lethargy, depression, malaise, anorexia, weight loss, muscle atrophy, and loss of lean body mass. In certain embodiments, the sign is anatomical, such as loss of muscle mass, which may be measured by ultrasound of muscle mass or by magnetic resonance imaging (MM). In particular embodiments, the symptom is a reduction in body weight, tumor-free body weight, liver weight, heart weight, muscle weight, or epididymal fat weight. In particular embodiments, the sign is a reduction in tibialis anterior muscle weight. The improvement, if measurable by percent, can be at least 1, 2, 3, 4, 5, 10, 15, 20, 25, 30, 40, 50, 60, 70, 80, or 90%, as compared to the absence of treatment with the iodide, e.g., NaI. Symptoms such as weakness, fatigue, pain, listlessness, depression, and malaise can be measured by techniques known in the art (e.g., using tests such as EORTC-global quality of life, the Beck Depression Inventory, the Zung Self-rating Depression Scale, the Center for Epidemiologic Studies-Depression Scale, the Hamilton Rating Scale for Depression, and patient self-reporting). Functional symptoms may also be determined or measured based on subject's answers to questionnaires directed to functional symptoms, sit to stand testing, six minute walk tests, stair ascent and descent testing, and strength (e.g., hand grip strength or leg extension strength), etc. For assessing anorexia, muscle mass, or lean body mass assessment, dual-emission X-ray absorptiometry scan (DEXA), bioelectrical impedance analysis (BIA), indirect calorimetry, nutrition diaries, and similar known methods can be used.

Methods disclosed herein may be used to treat, inhibit, or reduce the severity of any symptom of cardiotoxicity. Cardiotoxicity symptoms include but are not limited to cardiac dysfunction, which may be determined based on clinical symptoms or the use of echocardiogram or electrocardiogram (EKG). The presence of cardiotoxicity from chemotherapy has been traditionally assessed using clinical symptoms and decreases in left ventricular ejection fraction (LVEF). Examples of cardiotoxicity symptoms include but are not limited to left ventricular dysfunction (LVD): a decrease in cardiac LVEF that is either global or more severe in the septum; or a decline in LVEF of at least 10% to below 55%. In some embodiments, echocardiography is used to measure cardiac function and cardiotoxicity, e.g., subclinical cardiotoxicity. Cardiotoxicity can be measure as asymptomatic failure in the pumping of the heart that can progress to heart failure. It can present as abnormalities on electrocardiograms, irregular heartbeat, pericarditis-myocarditis syndrome (inflammation of the heart muscle or pericardium), or an increase in a brain peptide that is a marker of increased cardiac filling pressures. In some embodiments, nuclear imaging is used to measure cardiotoxicity. Nuclear imaging for cardiotoxicity may check cardiac function prior to and during treatment. A common nuclear medicine heart test is the radionuclide angiogram (RNA). This scan measures the amount of blood ejected from the ventricle with each heart beat (ejection fraction). For example, if the left ventricle ejects 60% of its blood volume with each beat, the LVEF is 0.6 (normal is 0.5 or greater). In some embodiments, an LVEF less than 0.5 is associated with cardiotoxicity. The improvement, if measureable by percent, can be at least 1, 2, 3, 4, 5, 10, 15, 20, 25, 30, 40, 50, 60, 70, 80, or 90%, as compared to the absence of treatment with the iodide, e.g., NaI.

Methods disclosed herein may extend the life of a subject being treated. The extension of survival of a mammalian subject with cachexia can be at least 10, 20, 30, 40, 50, 60, 70, 80, 90, 100, 150, or 200% over the expected lifespan of the subject. The expected lifespan of a subject with a particular disease associated with cachexia can be calculated by known methods, e.g., by averaging historical data. Expected survival times in cancer patients can be determined by known methods, e.g., as described in Llobera et al., Eur. J. Cancer, 36:2036, 2000 and McCusker et al., J. Chron. Dis., 37:377, 1984.

Dosing and Administration

In certain embodiments, the iodide, such as NaI, is provided to the subject in a pharmaceutical composition comprising a pharmaceutically acceptable carrier, diluent, or excipient. In certain embodiments, the pharmaceutical composition is a liquid, and in some embodiments, the pharmaceutical composition is a solid or semi-solid. In particular embodiments, a composition comprising the iodide comprises one or more of a reducing agent, a tonicity agent, a stabilizer, a surfactant, a lycoprotectant, a polyol, an antioxidant, or a preservative. In particular embodiments, the composition is a stable formulation formulated to maintain the iodide, e.g., NaI, in a reduced state. In some embodiments, at least 90% of the iodide in the composition is present in a reduced form for at least one hour, at least one week, at least one month, or at least six months when stored at room temperature.

In particular embodiments, the pharmaceutical composition comprising iodide, e.g., NaI, is provided to the subject prior to, during, or following the primary injury or disease, or the medical procedure. In certain embodiments, the subject is a mammal, e.g., a human.

A composition comprising the iodide compound, e.g., NaI, and a composition comprising a chemotherapeutic agent may be provided to the subject at the same time, at different times, or during an overlapping time period. In particular embodiments when both are used, the iodide compound and the chemotherapeutic agent are administered in the same composition or different compositions.

According to various embodiments of the methods of the present invention, a subject is provided with a composition of the invention, e.g., intravenously, intradermally, intraarterially, intraperitoneally, intralesionally, intracranially, intraarticularly, intraprostaticaly, intrapleurally, intratracheally, intranasally, intravitreally, intravaginally, intrarectally, topically, intratumorally, intramuscularly, intraperitoneally, intraocularly, subcutaneously, subconjunctival, intravesicularly, mucosally, intrapericardially, intraumbilically, orally, topically, locally, by injection, by infusion, by continuous infusion, by absorption, by adsorption, by immersion, by localized perfusion, via a catheter, or via a lavage. In particular embodiments, it is provided parenterally, e.g., intravenously, or by inhalation. “Parenteral” refers to any route of administration of a substance other than via the digestive tract. In specific embodiments, an iodide compound is provided to the subject by intravenous administration or infusion.

In certain embodiments, the pharmaceutical composition is provided to the subject orally or parenterally. For example the pharmaceutical composition may be provided to the subject as a bolus dose prior to the medical treatment associated with cachexia or cardiotoxicity, optionally wherein the bolus dose comprises less than or equal to about 10 mg/kg of halogen compound (e.g., NaI), optionally about 1.0 mg/kg or about 2 mg/kg. In other examples, the pharmaceutical composition is provided to the subject following the primary injury or disease or medical treatment. In some embodiment, multiple doses of the iodide compound (e.g., NaI) are provided to the subject. In particular embodiments, each dose comprises less than or equal to about 10 mg/kg of the iodide compound, optionally about 1.0 mg/kg or about 2.0 mg/kg of the iodide compound (e.g., NaI). In certain embodiments, multiple doses of the iodide compound are provided to the subject over a period of time, e.g., 4 hours, 8 hours, 12 hours, 1 day, 2 days, four days, 1 week, 2 weeks, 3 weeks, 1 month, 2 months, 4 months, 8 months, 1 year, or longer. In certain embodiments, the iodide compound (e.g., NaI) is provided to the subject as a continuous infusion, optionally prior to and/or during and/or following the primary injury or disease or medical treatment. In certain embodiments, less than about 100 mg/kg of iodide is provided to the subject over a period of time, e.g., 4 hours, 8 hours, 12 hours, 1 day, 2 days, four days, 1 week, 2 weeks, 3 weeks, 1 month, 2 months, 4 months, 8 months, 1 year, or longer. In various embodiments of methods of the present invention, a subject is exposed to a composition of the current invention for about, at least, at least about, or at most about 1, 2, 3, 4, 5, 6, 7, 8, 9, 10, 11, 12, 13, 14, 15, 16, 17, 18, 19, 20, 21, 22, 23, 24 hours, 1, 2, 3, 4, 5, 6, 7 days, 1, 2, 3, 4 weeks, 1, 2, 3, 4, 5, 6, 7, 8 or 9 months or more, and any range or combination therein.

In certain embodiments of methods of the invention, the iodide compound comprises iodide, e.g., NaI, and the effective amount is greater than or equal to about 150 μg, greater than or equal to about 300 μg, greater than or equal to about 500 μg, greater than or equal to about 1 mg, greater than or equal to about 2 mg, greater than or equal to about 5 mg, greater than or equal to about 10 mg, greater than or equal to about 15 mg, or greater than or equal to about 20 mg. In certain embodiments, the effective amount is 150 μg to 1000 mg, 300 μg to 1000 mg, 500 μg to 1000 mg, 1 mg to 1000 mg, 2 mg to 1000 mg, 5 mg to 1000 mg, 10 mg to 1000 mg, 150 μg to 100 mg, 300 μg to 100 mg, 500 μg to 100 mg, 1 mg to 100 mg, 2 mg to 100 mg, 5 mg to 100 mg, or 10 mg to 100 mg. In certain embodiments, the effective amount is 150 μg to 50 mg, 300 μg to 20 mg, 500 μg to 10 mg, 1 mg to 20 mg, 1 mg to 10 mg, or about 5 mg, about 10 mg, about 15 mg, or about 20 mg.

In particular embodiments, an effective amount of iodide compound or iodide is an amount at least or about two-fold, three-fold, four-fold, five-fold, six-fold, seven-fold, eight-fold, nine-fold, ten-fold, twelve-fold, fifteen-fold, twenty-fold, fifty-fold, 100-fold, 1,000-fold, 10,000-fold or 100,000-fold of the average daily recommended amounts as listed below. In particular embodiments, the effective amount of iodide compound, e.g., NaI, is an amount between about 100-fold to 2,000-fold or between about 500-fold to 1,500 fold the average daily recommended amounts as listed below for the indicated populations. In particular embodiments, the effective amount of iodide compound, e.g., NaI, is about 500-fold, about 1,000-fold, or about 1,500-fold the average daily recommended amounts as listed below for the indicated populations. In one embodiment, the effective amount of iodide compound, e.g., NaI, is about 1,000-fold the average daily recommended amounts as listed below, for the indicated populations. In particular embodiments, the effective amount of iodide is an amount between two-fold and twenty-fold, between five-fold and fifteen-fold, or between five-fold and ten-fold of the average daily recommended amounts of iodide as listed below. In certain embodiments, the iodide compound comprises iodide, e.g., NaI, and the effective amount is an amount that achieves about the same concentration or amount that is achieved by an effective amount of iodide that is at least or about two-fold, three-fold, four-fold, five-fold, six-fold, seven-fold, eight-fold, nine-fold, ten-fold, twelve-fold, fifteen-fold, or twenty-fold of the average daily recommended amounts as listed herein.

| | Recommended Amount¹ |
|---|---|
| Life Stage | (mcg) |
| Birth to 6 months | 110 |
| Infants 7-12 months | 130 |
| Children 1-8 years | 90 |
| Children 9-13 years | 120 |
| Teens 14-18 years | 150 |
| Adults | 150 |
| Pregnant teens and women | 220 |
| Breastfeeding teens and women | 290 |

¹NIH Office of Dietary Supplements Iodine Fact Sheet for Consumers, reviewed Jun. 24, 2011, obtained 2013.

In certain embodiments, the composition is provided to the subject in an amount sufficient to increase the blood concentration of the iodide compound, e.g., NaI at least five-fold, at least ten-fold, at least 50-fold, at least 100-fold, at least 500-fold, or at least 1000-fold for at least some time.

Furthermore, when administration of a composition according to the present invention is intravenous or by infusion, it is contemplated that the following parameters may be applied. A flow rate of about, at least about, or at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 gtts/min or μgtts/min, or any range derivable therein. In some embodiments, the amount of the composition is specified by volume, depending on the concentration of the iodide compound in the composition. An amount of time may be about, at least about, or at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60 minutes, 1, 2, 3, 4, 5, 6, 7, 8, 9, 10, 11, 12, 13, 14, 15, 16, 17, 18, 19, 20, 21, 22, 23, 24 hours, 1, 2, 3, 4, 5, 6, 7 days, 1, 2, 3, 4, 5 weeks, and/or 1, 2, 3, 4, 5, 6, 7, 8, 9, 10, 11, 12 months, or any range derivable therein.

Volumes of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 441, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 mls or any range therein, may be administered overall or in a single session.

In certain embodiments, a subject is administered an effective amount of an iodide, e.g., NaI, where the effective amount is between about 0.1 mg/kg to about 100 mg/kg, about 0.1 mg/kg to about 10 mg/kg, about 0.5 mg/kg to about 5 mg/kg, about 0.2 mg/kg, about 0.5 mg/kg, about 1.0 mg/kg, about 2.0 mg/kg, about 3.0 mg/kg, about 4.0 mg/kg, about 5.0 mg/kg, about 6.0 mg/kg, about 7.0 mg/kg, about 8.0 mg/kg, about 9.0 mg/kg or about 10 mg/kg. In particular embodiments of any of the methods of the present invention, a subject is treated with or contacted with an effective amount of a composition or compound of the present invention, wherein said effective amount of about 0.01 mg/kg to about 20 mg/kg, about 0.05 mg/kg to about 10 mg/kg, about 0.1 mg/kg to about 5 mg/kg, about 0.5 mg/kg to about 2 mg/kg, about 0.5 mg/kg to about 1 mg/kg, about 0.5 mg/kg, about 0.6 mg/kg, about 0.7 mg/kg, about 0.8 mg/kg, about 0.9 mg/kg, about 1.0 mg/kg, about 1.1 mg/kg or about 1.2 mg/kg. In certain embodiments, the composition comprises an iodide compound, e.g., NaI. In particular embodiments, any of these effective amounts is administered once per day. In other embodiments, any of these effective amounts is administered twice per day.

In certain embodiments, the effective amount is a dosage, e.g., a daily dosage, of 150 μg to 50 mg, 300 μg to 20 mg, 500 μg to 10 mg, 1 mg to 20 mg, 1 mg to 10 mg, or about 5 mg, about 10 mg, about 15 mg, or about 20 mg. In certain embodiments, the effective amount comprises less than or equal to 1000 mg, less than or equal to 800 mg, less than or equal to 700 mg, less than or equal to 500 mg, less than or equal to 250 mg, less than or equal to 200 mg, or less than or equal to 150 mg of the iodide compound. In certain embodiments, the effective amount is between about 100 mg and about 1000 mg (including any interval in this range), between about 150 mg and about 800 mg, between about 200 mg and about 700 mg, between about 250 mg and about 600 mg, between about 300 mg and about 500 mg, between about 350 mg and about 450 mg or between about 300 mg and about 700 mg of the iodide compound. In certain embodiments, the effective amount is about 200 mg, about 300 mg, about 400 mg, about 500 mg, about 600 mg, about 700 mg, about 800 mg, about 900 mg, or about 1000 mg of the iodide compound. In particular embodiments, the effective amount is the amount per day. In particular embodiments, any of these effective amounts is administered once per day. In other embodiments, any of these effective amounts is administered twice per day.

In particular embodiments, a cancer therapy, e.g., radiation therapy or chemotherapeutic agent, is provided to the subject according to the recommended dosage and administration route for the particular chemotherapeutic agent.

Iodide

The methods disclosed herein may be practiced using any source of iodide, such as NaI. Iodine (I), the second heaviest natural halogen, is the non-metal element with atomic number 53. Under standard pressure and temperature it exists as a solid diatomic 12 molecule. There are 34 iodine isotopes with known half-lives, said isotopes having mass numbers ranging from 108 to 144. Natural iodine, however, consists of one stable isotope, ¹²I.

In various embodiments, compositions and methods of the present invention comprise iodine. In particular embodiments, it is a reduced form of iodine, such as iodide. Certain embodiments may comprise an iodine-containing compound that is an iodide, or organoiodide.

In certain embodiments, the iodide is selected from or is provided to the subject as: sodium iodide, magnesium iodide, calcium iodide, hydrogen iodide, lithium iodide, silver iodide or zinc iodide. While sodium iodide is recited throughout as an illustrative iodide, it is understood that other sources of iodide disclosed herein may be substituted for sodium iodide in practicing the methods disclosed herein. In particular embodiments, methods disclosed herein are practiced using sodium iodide buffered with sodium chloride. In some embodiments, the iodide formulation or pharmaceutical composition comprises sodium iodide at a concentration of about 7.2 mg/mL balanced with sodium chloride to create a saline solution, with a pH in the range of about 7.0 to about 9.5.

In some embodiments, the iodide is selected from the non-limiting list of Aluminium iodide, Aluminium monoiodide, Ammonium iodide, Antimony triiodide, Arsenic diiodide, Arsenic triiodide, Barium iodide, Beryllium iodide, Bismuth(III) iodide, Boron triiodide, Cadmium iodide, Caesium iodide, Calcium iodide, Candocuronium iodide, Carbon tetraiodide, Cobalt(II) iodide, Coccinite, Copper(I) iodide, DiOC6, Diphosphorus tetraiodide, Dithiazanine iodide, Echothiophate, Einsteinium(III) iodide, Eschenmoser's salt, Ethylenediamine dihydroiodide, Gallium(III) iodide, GelGreen, GelRed, Germanium iodide, Gold monoiodide, Gold triiodide, Hydrogen iodide, Iodine oxide, Iodomethylzinc iodide, Iodosilane, Iron(II) iodide, Lead(II) iodide, Lithium iodide, Magnesium iodide, Manganese(II) iodide, Mercury(I) iodide, Mercury(II) iodide, Nickel(II) iodide, Nitrogen triiodide, Palladium(II) iodide, Phosphorus triiodide, Polyiodide, Potassium iodide, Potassium tetraiodomercurate(II), Propidium iodide, Rubidium iodide, Rubidium silver iodide, Samarium(II) iodide, Silicon tetraiodide, Silver iodide, Sodium iodide, Strontium iodide, Tellurium iodide, Tellurium tetraiodide, Terbium(III) iodide, Tetraethylammonium iodide, Thallium triiodide, Thallium(I) iodide, Thorium(IV) iodide, Tibezonium iodide, Tiemonium iodide, Tin(II) iodide, Tin(IV) iodide, Titanium tetraiodide, Triiodide, Trimethylsilyl iodide, Trimethylsulfoxonium iodide, Uranium pentaiodide, Uranium tetraiodide, Uranium triiodide, Vanadium(III) iodide, Zinc iodide, and Zirconium(IV) iodide.

In particular embodiments, the iodide is sodium iodide, potassium iodide, hydrogen iodide, calcium iodide, or silver iodide. In certain embodiments, the iodide is sodium iodide.

In some embodiments, iodide is an organoiodide comprising one or more compounds from the non-limiting list of ²⁵I-NBF, ²⁵I-NBMD, ²⁵I-NBOH, ²⁵I-NBOMe, 2C-I, 5, 5-I-R91150, Acetrizoic acid, Adipiodone, Adosterol, Altropane, AM-1241, AM-2233, AM-630, AM-679 (cannabinoid), AM-694, AM251, Amiodarone, Benziodarone, Bromoiodomethane, Budiodarone, Butyl iodide, Carbon tetraiodide, Chiniofon, Chloroiodomethane, Clioquinol, Diatrizoic acid, Diiodohydroxypropane, Diiodohydroxyquinoline, Diiodomethane, 2,5-Dimethoxy-4-iodoamphetamine, Domiodol, Erythrosine, Ethyl iodide, Ethyl iodoacetate, Fialuridine, Fluoroiodomethane, Haloprogin, Herapathite, IAEDANS, Ibacitabine, IDNNA, Idoxifene, Idoxuridine, Iniparib, Iobenguane, Iobenzamic acid, Iobitridol, Iocarmic acid, Iocetamic acid, Iodamide, Iodixanol, Iodoacetamide, Iodoacetic acid, Para-Iodoamphetamine, Iodobenzamide, Iodobenzene, 2-Iodobenzoic acid, 19-Iodocholesterol, Iodocyanopindolol, Iodoform, 1-Iodomorphine, Iodophenol, Iodophenpropit, 4-Iodopropofol, Iodopropynyl butylcarbamate, Iodotrifluoroethylene, Iodoxamic acid, 2-Iodoxybenzoic acid, Iofetamine (1231), Ioflupane (1231), Ioglicic acid, Ioglycamic acid, Iomazenil, Iomeprol, Iopamidol, Iopanoic acid, Iopentol, Iopromide, Iopydol, Iotrolan, Iotroxic acid, Ioversol, Ioxaglic acid, Ioxilan, Ipodate sodium, Isopropyl iodide, Methiodal, Methyl iodide, Metrizamide, Metrizoic acid, Pentafluoroethyl iodide, Plakohypaphorine, N-Propyl iodide, Propyliodone, Rafoxanide, Rose bengal, RTI-121, RTI-229, RTI-353, RTI-55, SB-258,585, Sodium acetrizoate, Tiratricol, Trifluoroiodomethane, and Tyropanoic acid.

In particular embodiments, the iodide is an organoiodide. Organoiodine compounds are organic compounds that contain one or more carbon-iodine bonds. Almost all organoiodine compounds feature iodide connected to one carbon center. These are usually classified as derivatives of I⁻. Some organoiodine compounds feature iodine in higher oxidation states. Organoiodine compounds, often used as disinfectants or pesticides, include, e.g., iodoform (CHIS), methylene iodide (CH₂I₂), and methyl iodide (CH₃I). In particular embodiment, the organoiodide is a polyiodoorganic compound. Polyiodoorganic compounds are sometimes employed as X-ray contrast agents, in fluoroscopy, a type of medical imaging. A variety of such polyiodoorganic compounds are available commercially; many are derivatives of 1,3,5-triiodobenzene and contain about 50% by weight iodine. In certain embodiments, the agent is soluble in water, non-toxic and/or readily excreted. A representative reagent is Ioversol, which has water-solubilizing diol substituents. Other organoiodine compounds include but are not limited to the two thyroid hormones thyroxine (“T4”) and triiodothyronine (“T3”). Marine natural products are rich sources of organoiodine compounds, including the recently discovered plakohypaphorines from the sponge Plakortis simplex.

The present invention also includes the use of compounds, e.g., drug compounds, into which an iodine is incorporated. For example, an iodine may be incorporated into existing drugs such as N-acetyl cysteine, standard pain relievers, and non-steroidal anti-inflammatory drugs, such as, e.g., aspirin, ibuprofen and naproxen. Most NSAIDs act as nonselective inhibitors of the enzyme cyclooxygenase (COX), inhibiting both the cyclooxygenase-1 (COX-1) and cyclooxygenase-2 (COX-2) isoenzymes.

In certain embodiments, the iodide is a polyiodide. The polyiodides are a class of polyhalogen anions composed of entirely iodine atoms. The most common and simplest member is the triiodide ion, Other known, larger polyiodides include [I₄]²⁻, [I₅]⁻, [I₇]⁻, [I₈]²⁻, [I₉]⁻, [I₁₀]²⁻, [I₁₀]⁴⁻, [I₁₂]²⁻, [I₁₃]³⁻, [I₁₆]²⁻, [I₂₂]⁴⁻, [I₂₆]³⁻, [I₂₆]⁴⁻, [I₂₈]⁴⁻ and [I₂₉]³⁻. One example of a polyiodide is Lugol's iodine, also called Lugol's solution. Lugol's solution is commercially available in different potencies of 1%, 2%, or 5% Iodine. The 5% solution consists of 5% (wt/v) iodine (I₂) and 10% (wt/v) potassium iodide (KI) mixed in distilled water and has a total iodine content of 130 mg/mL. Potassium iodide renders the elementary iodine soluble in water through the formation of the triiodide (I⁻³) ion. Other names for Lugol's solution are I₂KI (iodine-potassium iodide); Markodine, Strong solution (Systemic); and Aqueous Iodine Solution BCP. Examples of polyiodides, including their ions and counter-cations are shown in Table 1.

TABLE 1
Polyiodides

| Anion | Counter-cation |
|---|---|
| [I₃]⁻ | Cs⁺ |
| [I₄]²⁻ | [Cu(NH₃)₄]²⁺ |
| [I₅]⁻ | [EtMe₃N]⁺ |
| | [EtMePh₂N]⁺ |
| [I₇]⁻ | [Ag(18aneS₆)]⁺ |
| [I₈]²⁻ | [Ni(phen)₃]²⁺ |
| [I₉]− | [Me₂ⁱPrPhN]⁺ |
| | [Me₄N]⁺ |
| [I₁₀]²⁻ | [Cd(12-crown-4)₂]²⁺ |
| [I₁₁]³⁻ | [(16aneS₄)PdIPd(16aneS₄)]³⁺ |
| [I₁₂]²⁻ | [Ag₂(15aneS₅)₂]²⁺ |
| | [Cu(Dafone)₃]²⁺ |
| [I₁₃]³⁻ | [Me₂Ph₂N]⁺ |
| [I₁₆]²⁻ | [Me₂Ph₂N]⁺ |
| | [ⁱPrMe₂PhN]⁺ |
| [I₂₂]⁴⁻ | [MePh₃P]⁺ |
| [I₂₆]³⁻ | [Me₃S]⁺ |
| [I₂₆]⁴⁻ | DMFc⁺ |
| [I₂₉]³⁻ | Cp₂Fe |
| [I₂₂]⁴⁻ | [MePh₃P]⁺ |
| [I₂₆]³⁻ | [Me₃S]⁺ |
| [I₂₆]⁴⁻ | DMFc⁺ |
| [I₂₉]³⁻ | Cp₂Fe |
| [I₂₂]⁴⁻ | [MePh₃P]⁺ |
| [I₂₆]³⁻ | [Me₃S]⁺ |
| [I₂₆]⁴⁻ | DMFc⁺ |

In one embodiment, the iodide is a tincture of iodine solutions, which comprises or consists of elemental iodine, and iodide salts dissolved in water and alcohol.

In one embodiment, the iodide is an oil-infused iodide or iodine oil infusion.

Particular embodiments of the present invention relate to a reduced form of an iodide compound. Many acceptable means of reduction of iodine are possible and known to one skilled in the art. Examples of reduced forms of iodine compounds include iodide, wherein the iodine has a valency of −1., including salt forms, such as Nat Non-limiting examples of reduction methods include chemical reduction with electropositive elemental metals (such as lithium, sodium, magnesium, iron, zinc, and aluminum, e.g.), hydride transfer reagents (such as NaBH₄ and LiAIH₄ e/g), or the use of hydrogen gas with a palladium, platinum, or nickel catalyst.

A particular embodiment of the present invention relates to the administration of an iodide (e.g., NaI), to a mammalian subject, in a composition, concentration or formulation that is not significantly toxic to said mammals, e.g., a pharmaceutical composition. In particular embodiments, an iodide known to be toxic to a mammalian subject is excluded from the present invention. Thus, in particular embodiments, parenterally administered potassium iodide is excluded from the present invention. It is further contemplated that some embodiments may comprise the administration of more than one of said iodide compounds to said mammal, either simultaneously or separately, such that the combination of said compounds that are not individually significantly toxic are also not significantly toxic when combined.

Other compounds comprising an iodide may also be used according to methods of and/or included in compositions of the present invention. In some embodiments, said iodide compound is a commercially available substance. In certain embodiments, said commercially available substances may include radiological contrast agents, topical iodine preparations, solutions, or drugs. In certain embodiments, said commercially available substance comprises iodide, and may be selected from the non-limiting list of Diatrizoate, Ipanoic acid, Ipodate, Iothalamate, Metrizamide, Diatrozide, Diiodohydroxyquinolone, Iodine tincture, Povidone iodine, Iodochlorohydroxyquinolone, Iodoform gauze, Saturated potassium iodide (S SKI), Lugol solution, Iodinated glycerol, Echothiopate iodide, Hydriodic acid syrup, Calcium iodide, Amiodarone, Expectorants, Vitamins containing iodine, Iodochlorohydroxyquinolone, Diiodohydroxyquinolone, Potassium iodide, Benziodarone, Isopropamide iodide, levothyroxine, and Erythrosine.

In various embodiments, the iodide, e.g., NaI, used according to the disclosed methods is present in a formulation or pharmaceutical composition, e.g., a pharmaceutical compositions comprising an iodide, e.g., NaI, and one or more pharmaceutically acceptable carriers, diluents or excipients, e.g., a buffer. Further, any of the compositions may comprise one or more of a buffer, a reducing agent, a tonicity agent, a stabilizer, a surfactant, a lycoprotectant, a polyol, an antioxidant, or a preservative. In particular embodiments, any of the compositions described herein comprise glutathione. In particular embodiments, compositions may comprise one or more solvents. In particular embodiments, the solvent is water. In particular embodiments, the solvent is a phosphate-buffered saline. In particular embodiments, the composition further comprises one or more additional active agents, e.g., a chemotherapeutic agent or another agent used to treat or prevent cachexia, including but not limited to any disclosed herein.

In certain embodiments, the pharmaceutical compositions comprise a reduced form of iodine, such as iodide. In particular embodiments, the compound containing a reduced form of iodine is NaI. In particular embodiments, the compositions are formulated to maintain the iodide in a reduced form when stored over a period of time. Thus, the compositions may be stable compositions of reduced forms of iodide or salts or precursors thereof, whose effectiveness as a therapeutic may normally be compromised during manufacture and storage, as a result of oxidation reactions that produce oxidation products.

In certain embodiments of the compositions, a composition is considered stable, i.e., a stable composition, if at least 90% of the iodide in the composition is present in reduced form for at least one hour either when stored at room temperature, 4° C., 25° C., 40° C. or 50° C. In related embodiments, a composition is considered stable if at least 70%, at least 80%, at least 91%, at least 92%, at least 93%, at least 94%, at least 95%, at least 96%, at least 97%, at least 98%, or at least 99% of the halogen compound in the composition is present in reduced form for at least one hour either when stored at room temperature or when stored at 4° C. In certain embodiments of the stable compositions, at least 90% of the halogen compound in said composition is present in said reduced form for at least one day, at least one week, at least one month, at least two months, at least four months, at least six months, or at least one year, either when stored at room temperature or when stored at 4° C., 25° C., 40° C. or 50° C. In related embodiments, at least 70%, at least 80%, at least 91%, at least 92%, at least 93%, at least 94%, at least 95%, at least 96%, at least 97%, at least 98%, or at least 99% of the halogen compound in the stable composition is present in said reduced form for at least one day, at least one week, at least one month, at least two months, at least four months, at least six months, or at least one year, either when stored at room temperature or when stored at 4° C. In particular embodiments, at least 98% of the halogen compound in the stable composition is present in said reduced form for at least one month or at least six months when stored at 4° C. In related embodiments, at least 70%, at least 80%, at least 91%, at least 92%, at least 93%, at least 94%, at least 95%, at least 96%, at least 97%, at least 98%, or at least 99% of the halogen compound in the stable composition is present in said reduced form for at least one day, at least one week, at least one month, at least two months, at least four months, at least six months, or at least one year, either when stored at room temperature or when stored at room temperature or 25° C. In particular embodiments, at least 98% of the halogen compound in the stable composition is present in said reduced form for at least one month or at least six months when stored at room temperature or 25° C. In related embodiments, at least 70%, at least 80%, at least 91%, at least 92%, at least 93%, at least 94%, at least 95%, at least 96%, at least 97%, at least 98%, or at least 99% of the halogen compound in the stable composition is present in said reduced form for at least one day, at least one week, at least one month, at least two months, at least four months, at least six months, or at least one year, either when stored at room temperature or when stored at 40° C. or 50° C. In particular embodiments, at least 98% of the halogen compound in the stable composition is present in said reduced form for at least one month or at least six months when stored at 40° C. or 50° C.

In certain embodiments, a composition of the invention may be formulated in form suitable for oral or parenteral administration

In various embodiments, the composition is a liquid pharmaceutical composition, while in other embodiments, the composition is a solid or powder, or is dried, lyophilized, or freeze-dried. In particular embodiments, the present invention relates to a stable liquid composition comprising iodide, wherein the stable liquid composition comprises less than 1% of any of the following oxidation products of iodide (−1 oxidation state): hypoiodite (+1 oxidation state), iodite (+3 oxidation state), iodate (+5 oxidation state), or periodate (+7 oxidation state). In particular embodiments, the stable liquid composition comprising iodide comprises less than 1% iodine (I₂).

In some embodiments, the concentration of iodide, e.g., NaI, present in a composition of the present invention is about 0.0001 mM to about 100 M, about 0.0005 mM to about 50 M, about 0.001 mM to about 10 M, about 0.001 mM to about 5 M, about 0.001 mM to about 1 M, about 0.005 mM to about 10 M, about 0.005 mM to about 5 M, about 0.005 mM to about 1 M, about 0.005 mM to about 0.5 M, about 0.01 mM to about 10 M, about 0.01 mM to about 5 M, about 0.01 mM to about 2 M, about 0.1 mM to about 1 M, about 0.1 mM to about 0.5 M, about 0.5 mM to about 5 M, about 0.5 mM to about 2 M, about 0.5 mM to about 1 M, about 0.5 mM to about 0.5 M, about 1 mM to about 5 M, about 1 mM to about 2 M, about 1 mM to about 1 M, about 1 mM to about 0.5 M, about 5 mM to about 5 M, about 5 mM to about 2 M, about 5 mM to about 1 M, about 5 mM to about 0.5 M, about 5 mM to about 0.25 M, about 10 mM to about 1 M, about 10 mM to about 0.5 M, about 10 mM to about 0.25 M, or about 10 mM, about 50 mM about 100 mM, or about 200 mM.

In particular embodiments, the pH of a composition of the present invention is in the range of (3.0-12.0), while in other embodiments, the pH is in the range of (5.0-9.0). The pH of the pharmaceutical composition may be adjusted to a physiologically compatible range. For example, in one embodiment, the pH of the stable composition is in the range of 6.5-8.5. In other embodiments, the compositions of the present invention have a pH in the range of 7.5-8.5 or 7.4-9.0.

In particular embodiments, oxygen is present in the composition at a concentration of less than 3 μM, less than 1 μM, less than 0.1 μM, less than 0.01 μM, or less than 0.001 μM.

In certain embodiments, the compositions of the present invention may further comprise a limited amount of oxidation products. Oxidation products that may be present in various embodiments of the present invention include, but are not limited to, iodine and iodate. In various embodiments, one or more of these oxidation products is present in a composition in an amount less than 10%, less than 5.0%, less than 2.0%, less than 1.0%, less than 0.5%, less than 0.2%, less than 0.1%, less than 0.05%, or less than 0.01% (w/v) of the total halogen compound in the composition.

In one embodiment, a composition has an osmolarity in the range of 200-400 mOsmol/L. NaCl may be used as an excipient to adjust osmolality.

In certain embodiments, the composition has a pH in the range of 6.5 to 8.5 and has an oxygen content of less than or equal to 5 μM for 3 months when stored within a temperature range of 23°-27° or 6 months when stored at a temperature range of)(23°-27°. In one embodiment, the composition has an osmolarity in the range of 250-330 mOsmol/L. It may be isotonic or near isotonic.

Tumors and Cancer

In some embodiments, the subject has been diagnosed and/or is being treated for a tumor or cancer. In particular embodiments, the cancer is carcinoma, sarcoma, melanoma, lymphoma or leukemia. In other embodiments, the cancer is a hematologic malignancy. In some embodiments, the cancer is leukemia (e.g., chronic lymphocytic leukemia), lymphoma (e.g., non-Hodgkin's lymphoma), or multiple myeloma. In other embodiments, the cancer is a solid tumor.

In some variations, the cancer is small lymphocytic lymphoma, non-Hodgkin's lymphoma, indolent non-Hodgkin's lymphoma (iNHL), refractory non-Hodgkin's lymphoma rNHL, mantle cell lymphoma, follicular lymphoma, lymphoplasmacytic lymphoma, marginal zone lymphoma, immunoblastic large cell lymphoma, lymphoblastic lymphoma, Splenic marginal zone B-cell lymphoma (+/−villous lymphocytes), nodal marginal zone lymphoma (+/−monocytoid B-cells), extranodal marginal zone B-cell lymphoma of mucosa-associated lymphoid tissue type, cutaneous T-cell lymphoma, extranodal T-cell lymphoma, anaplastic large cell lymphoma, angioimmunoblastic T-cell lymphoma, mycosis fungoides, B-cell lymphoma, diffuse large B-cell lymphoma, Mediastinal large B-cell lymphoma, Intravascular large B-cell lymphoma, Primary effusion lymphoma, small non-cleaved cell lymphoma, Burkitt's lymphoma, multiple myeloma, plasmacytoma, acute lymphocytic leukemia, T-cell acute lymphoblastic leukemia, B-cell acute lymphoblastic leukemia, B-cell prolymphocytic leukemia, acute myeloid leukemia, chronic lymphocytic leukemia, juvenile myelomonocytic leukemia, minimal residual disease, hairy cell leukemia, primary myelofibrosis, secondary myelofibrosis, chronic myeloid leukemia, myelodysplastic syndrome, myeloproliferative disease, or Waldestrom's macroglobulinemia.

In some embodiments, the cancer is pancreatic cancer, urological cancer, bladder cancer, (e.g., urothelial bladder cancer, UBC), colorectal cancer, colon cancer, breast cancer, prostate cancer, renal cancer, hepatocellular cancer, thyroid cancer, gall bladder cancer, lung cancer (e.g., non-small cell lung cancer, small-cell lung cancer), ovarian cancer, cervical cancer, gastric cancer, endometrial cancer, esophageal cancer, head and neck cancer, melanoma, neuroendocrine cancer, CNS cancer, brain tumors (e.g., glioma, anaplastic oligodendroglioma, adult glioblastoma multiforme, and adult anaplastic astrocytoma), bone cancer, soft tissue sarcoma, retinoblastomas, neuroblastomas, peritoneal effusions, malignant pleural effusions, mesotheliomas, Wilms tumors, trophoblastic neoplasms, hemangiopericytomas, Kaposi's sarcomas, myxoid carcinoma, round cell carcinoma, squamous cell carcinomas, esophageal squamous cell carcinomas, oral carcinomas, cancers of the adrenal cortex, or ACTH-producing tumors.

Particular examples of cancer include, but are not limited to, carcinoma, lymphoma, blastoma (including medulloblastoma and retinoblastoma), sarcoma (including liposarcoma and synovial cell sarcoma), neuroendocrine tumors (including carcinoid tumors, gastrinoma, and islet cell cancer), mesothelioma, schwannoma (including acoustic neuroma), meningioma, adenocarcinoma, melanoma, and leukemia or lymphoid malignancies. More particular examples of such cancers include bladder cancer (e.g., urothelial bladder cancer (e.g., transitional cell or urothelial carcinoma, non-muscle invasive bladder cancer, muscle-invasive bladder cancer, and metastatic bladder cancer) and non-urothelial bladder cancer), squamous cell cancer (e.g., epithelial squamous cell cancer), lung cancer including small-cell lung cancer (SCLC), non-small cell lung cancer (NSCLC), adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, hepatoma, breast cancer (including metastatic breast cancer), colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, Merkel cell cancer, mycoses fungoids, testicular cancer, esophageal cancer, tumors of the biliary tract, as well as head and neck cancer and hematological malignancies. In some embodiments, the cancer is triple-negative metastatic breast cancer, including any histologically confirmed triple-negative (ER−, PR−, HER2−) adenocarcinoma of the breast with locally recurrent or metastatic disease (where the locally recurrent disease is not amenable to resection with curative intent).

Cancer Therapy.

In particular embodiments, the subject being treated has cachexia or cardiotoxicity associated with or resulting from treatment of a tumor or cancer. In particular embodiments, the cancer therapy comprises radiation therapy or chemotherapy using any of a variety of chemotherapeutic agents, including but not limited to those disclosed herein.

In certain embodiments, the cachexia or cardiotoxicity being treated is associated with or results from radiation therapy, including but not limited to any type disclosed herein. Radiation therapy uses high doses of radiation to kill cancer cells and shrink tumors. In some embodiments, the radiation therapy is external beam radiation therapy, while in some embodiments, the radiation therapy is internal radiation therapy, in which the source of the radiation is placed inside the body. The radiation source can be solid or liquid. Internal radiation therapy with a solid source is called brachytherapy. In this type of treatment, seeds, ribbons, or capsules that contain a radiation source are placed in the body, in or near the tumor.

In certain embodiments, the cachexia or cardiotoxicity being treated is associated with or results from treatment with a chemotherapeutic agent, including but not limited to any type disclosed herein. A “chemotherapeutic agent” is a chemical compound useful in the treatment of cancer, and include various types of compounds, including, e.g., small molecules, antibodies, and nucleic acids. Any of those disclosed herein and other may be used according to the methods disclosed herein. In particular embodiments, the cancer therapy, e.g., radiation therapy or chemotherapeutic agent, is associated with or can result in cachexia or cardiotoxicity. It has been reported that several chemotherapeutics, including but not limited to anthracycline antibiotics (e.g., doxorubicin), cisplatin, cyclophosphamide, antibodies (e.g., trastuzumab), CPT-11, paclitaxel, adriamycin, etoposide, Folfiri (5-fluorouracil, irinotecan, and leucovorin), and methotrexate can cause cachexia and/or cardiotoxicity.

Chemotherapeutic agents may be categorized by their mechanism of action into, for example, the following groups: anti-metabolites/anti-cancer agents such as pyrimidine analogs floxuridine, capecitabine, and cytarabine; purine analogs, folate antagonists, and related inhibitors; antiproliferative/antimitotic agents including natural products such as vinca alkaloid (vinblastine, vincristine) and microtubule inhibitors such as taxane (paclitaxel, docetaxel), vinblastin, nocodazole, epothilones, vinorelbine (NAVELBINE), and epipodophyllotoxins (etoposide, teniposide); DNA damaging agents such as actinomycin, amsacrine, busulfan, carboplatin, chlorambucil, cisplatin, cyclophosphamide, dactinomycin, daunorubicin, doxorubicin, epirubicin, iphosphamide, melphalan, merchlorethamine, mitomycin, mitoxantrone, nitrosourea, procarbazine, taxol, taxotere, teniposide, etoposide, and triethylenethiophosphoramide; antibiotics such as dactinomycin, daunorubicin, doxorubicin, idarubicin, anthracyclines, mitoxantrone, bleomycins, plicamycin (mithramycin), and mitomycin; enzymes such as L-asparaginase which systemically metabolizes L-asparagine and deprives cells which do not have the capacity to synthesize their own asparagine; antiplatelet agents; antiproliferative/antimitotic alkylating agents such as nitrogen mustards cyclophosphamide and analogs (melphalan, chlorambucil, hexamethylmelamine, and thiotepa), alkyl nitrosoureas (carmustine) and analogs, streptozocin, and triazenes (dacarbazine); antiproliferative/antimitotic antimetabolites such as folic acid analogs (methotrexate); platinum coordination complexes such as cisplatin, oxiloplatinim, and carboplatin), procarbazine, hydroxyurea, mitotane, and aminoglutethimide; hormones and hormone analogs such as estrogen, tamoxifen, goserelin, bicalutamide, and nilutamide, and aromatase inhibitors such as letrozole and anastrozole; anticoagulants such as heparin, synthetic heparin salts, and other inhibitors of thrombin; fibrinolytic agents such as tissue plasminogen activator, streptokinase, urokinase, aspirin, dipyridamole, ticlopidine, and clopidogrel; antimigratory agents; antisecretory agents such as breveldin; immunosuppressives such as tacrolimus, sirolimus, azathioprine, and mycophenolate; compounds (TNP-470, genistein) and growth factor inhibitors (vascular endothelial growth factor inhibitors and fibroblast growth factor inhibitors); angiotensin receptor blockers, nitric oxide donors; antisense oligonucleotides; antibodies such as trastuzumab and rituximab; cell cycle inhibitors and differentiation inducers such as tretinoin; inhibitors including topoisomerase inhibitors such as doxorubicin, daunorubicin, dactinomycin, eniposide, epirubicin, etoposide, idarubicin, irinotecan, mitoxantrone, topotecan, and irinotecan, and corticosteroids such as cortisone, dexamethasone, hydrocortisone, methylprednisolone, prednisone, and prednisolone; growth factor signal transduction kinase inhibitors; dysfunction inducers; toxins such as Cholera toxin, ricin, Pseudomonas exotoxin, Bordetella pertussis adenylate cyclase toxin, diphtheria toxin, and caspase activators; and chromatin.

Further examples of chemotherapeutic agents include: alkylating agents such as thiotepa and cyclophosphamide; alkyl sulfonates such as busulfan, improsulfan, and piposulfan; aziridines such as benzodopa, carboquone, meturedopa, and uredopa; emylerumines and memylamelamines including alfretamine, triemylenemelamine, triethylenephosphoramide, triethylenethiophosphoramide, and trimemylolomelamine; acetogenins, especially bullatacin and bullatacinone; a camptothecin, including synthetic analog topotecan; bryostatin; callystatin; CC-1065, including its adozelesin, carzelesin, and bizelesin synthetic analogs; cryptophycins, particularly cryptophycin 1 and cryptophycin 8; dolastatin; duocarmycin, including the synthetic analogs KW-2189 and CBI-TMI; eleutherobin; pancratistatin; a sarcodictyin; spongistatin; nitrogen mustards such as chlorambucil, chlornaphazine, cyclophosphamide, estramustine, ifosfamide, mechlorethamine, mechlorethamine oxide hydrochloride, melphalan, novembichin, phenesterine, prednimustine, trofosfamide, and uracil mustard; nitrosoureas such as carmustine, chlorozotocin, foremustine, lomustine, nimustine, and ranimustine; antibiotics such as the enediyne antibiotics (e.g., calicheamicin, especially calicheamicin gammall and calicheamicin phill), dynemicin including dynemicin A, bisphosphonates such as clodronate, an esperamicin, neocarzinostatin chromophore and related chromoprotein enediyne antibiotic chromomophores, aclacinomycins, actinomycin, authramycin, azaserine, bleomycins, cactinomycin, carabicin, carrinomycin, carzinophilin, chromomycins, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin, and deoxydoxorubicin), epirubicin, esorubicin, idarubicin, marcellomycin, mitomycins such as mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, and zombicin; anti-metabolites such as methotrexate and 5-fluorouracil (5-FU); folic acid analogs such as demopterin, methotrexate, pteropterin, and trimetrexate; purine analogs such as fludarabine, 6-mercaptopurine, thiamiprine, and thioguanine; pyrimidine analogs such as ancitabine, azacitidine, 6-azauridine, carmofur, cytarabine, dideoxyuridine, doxifluridine, enocitabine, and floxuridine; androgens such as calusterone, dromostanolone propionate, epitiostanol, mepitiostane, and testolactone; anti-adrenals such as aminoglutethimide, mitotane, and trilostane; folic acid replinishers such as frolinic acid; trichothecenes, especially T-2 toxin, verracurin A, roridin A, and anguidine; taxoids such as paclitaxel and docetaxel; platinum analogs such as cisplatin and carboplatin; aceglatone; aldophosphamide glycoside; aminolevulinic acid; eniluracil; amsacrine; hestrabucil; bisantrene; edatraxate; defofamine; demecolcine; diaziquone; elformthine; elliptinium acetate; an epothilone; etoglucid; gallium nitrate; hydroxyurea; lentinan; leucovorin; lonidamine; maytansinoids such as maytansine and ansamitocins; mitoguazone; mitoxantrone; mopidamol; nitracrine; pentostatin; phenamet; pirarubicin; losoxantrone; fluoropyrimidine; folinic acid; podophyllinic acid; 2-ethylhydrazide; procarbazine; polysaccharide-K (PSK); razoxane; rhizoxin; sizofiran; spirogermanium; tenuazonic acid; triaziquone; 2,2′,2″-tricUorotriemylamine; urethane; vindesine; dacarbazine; mannomustine; mitobronitol; mitolactol; pipobroman; gacytosine; arabinoside (“Ara-C”); cyclophosphamide; thiopeta; chlorambucil; gemcitabine; 6-thioguanine; mercaptopurine; methotrexate; vinblastine; platinum; etoposide (VP-16); ifosfamide; mitroxantrone; vancristine; vinorelbine (NAVELBINE®); novantrone; teniposide; edatrexate; daunomycin; aminopterin; xeoloda; ibandronate; CPT-11; topoisomerase inhibitor RFS 2000; difluoromethylornithine (DFMO); retinoids such as retinoic acid; capecitabine; FOLFIRI (fluorouracil, leucovorin, and irinotecan); and pharmaceutically acceptable salts, acids, or derivatives of any of the above.

Examples of chemotherapeutic agents include alkylating agents such as thiotepa and CYTOXANO cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); delta-9-tetrahydrocannabinol (dronabinol, MARINOL®); beta-lapachone; lapachol; cochicines; betulinic acid; a camptothecin (including the synthetic analogue topotecan (HYCAMTIN®), CPT-11 (irinotecan, CAMPTOSAR®), acetylcamptothecin, scopolectin, and 9-aminocamptothecin); bryostatin; callystatin; CC-1065 (including its adozelesin, carzelesin and bizelesin synthetic analogues); podophyllotoxin; podophyllinic acid; teniposide; cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosoureas such as carmustine, chlorozotocin, fotemustine, lomustine, nimustine, and ranimnustine; antibiotics such as the enediyne antibiotics (e.g., calicheamicin, especially calicheamicin.gamma.1I and calicheamicin.omega.1I dynemicin, including dynemicin A; an esperamicin; as well as neocarzinostatin chromophore and related chromoprotein enediyne antiobiotic chromophores, aclacinomysins, actinomycin, authramycin, azaserine, bleomycins, cactinomycin, carabicin, carminomycin, carzinophilin, chromomycin, dactinomycin, daunorubicin, detorubicin, 6-diazo-5-oxo-L-norleucine, ADRIAMYCINO doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfornithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; 2-ethylhydrazide; procarbazine; PSK® polysaccharide complex; razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine (ELDISINEO, FILDESIN®); dacarbazine; mannomustine; mitobronitol; mitolactol; pipobroman; gacytosine; arabinoside (“Ara-C”); thiotepa; taxoids, for example taxanes including TAXOL® paclitaxel, ABRAXANE™ Cremophor-free, albumin-engineered nanoparticle formulation of paclitaxel, and TAXOTEREO docetaxel; chlorambucil; gemcitabine (GEMZAR®); 6-thioguanine; mercaptopurine; methotrexate; platinum or platinum-based chemotherapy agents and platinum analogs, such as cisplatin, carboplatin, oxaliplatin (ELOXATIN™), satraplatin, picoplatin, nedaplatin, triplatin, and lipoplatin; vinblastine (VELBAN®); platinum; etoposide (VP-16); ifosfamide; mitoxantrone; vincristine (ONCOVIN®); oxaliplatin; leucovovin; vinorelbine (NAVELBINE®); novantrone; edatrexate; daunomycin; aminopterin; ibandronate; topoisomerase inhibitor RFS 2000; difluorometlhylornithine (DMF®); retinoids such as retinoic acid; capecitabine (XELODA®); pharmaceutically acceptable salts, acids or derivatives of any of the above; as well as combinations of two or more of the above such as CHOP, an abbreviation for a combined therapy of cyclophosphamide, doxorubicin, vincristine, and prednisolone, and FOLFOX, an abbreviation for a treatment regimen with oxaliplatin (ELOXATIN™) combined with 5-FU and leucovorin. Additional chemotherapeutic agents include the cytotoxic agents useful as antibody drug conjugates, such as maytansinoids (DM1, for example) and the auristatins MMAE and MMAF, for example.

“Chemotherapeutic agents” also include “anti-hormonal agents” or “endocrine therapeutics” that act to regulate, reduce, block, or inhibit the effects of hormones that can promote the growth of cancer, and are often in the form of systemic, or whole-body treatment. They may be hormones themselves. Examples include anti-estrogens and selective estrogen receptor modulators (SERMs), including, for example, tamoxifen (including NOLVADEX® tamoxifen), EVISTA® raloxifene, droloxifene, 4-hydroxytamoxifen, trioxifene, keoxifene, LY117018, onapristone, and FARESTONO toremifene; anti-progesterones; estrogen receptor down-regulators (ERDs); agents that function to suppress or shut down the ovaries, for example, leutinizing hormone-releasing hormone (LHRH) agonists such as LUPRON® and ELIGARD® leuprolide acetate, goserelin acetate, buserelin acetate and tripterelin; other anti-androgens such as flutamide, nilutamide and bicalutamide; and aromatase inhibitors that inhibit the enzyme aromatase, which regulates estrogen production in the adrenal glands, such as, for example, 4(5)-imidazoles, aminoglutethimide, MEGASE® megestrol acetate, AROMASIN® exemestane, formestanie, fadrozole, RIVISOR® vorozole, FEMARA® letrozole, and ARIMIDEX® anastrozole. In addition, such definition of chemotherapeutic agents includes bisphosphonates such as clodronate (for example, BONEFOS® or OSTAC®), DIDROCAL® etidronate, NE-58095, ZOMETA® zoledronic acid/zoledronate, FOSAMAX® alendronate, AREDIA® pamidronate, SKELID® tiludronate, or ACTONEL® risedronate; as well as troxacitabine (a 1,3-dioxolane nucleoside cytosine analog); antisense oligonucleotides, particularly those that inhibit expression of genes in signaling pathways implicated in aberrant cell proliferation, such as, for example, PKC-alpha, Raf, H-Ras, and epidermal growth factor receptor (EGFR); vaccines such as THERATOPE® vaccine and gene therapy vaccines, for example, ALLOVECTIN® vaccine, LEUVECTIN® vaccine, and VAXID® vaccine; LURTOTECANO topoisomerase 1 inhibitor; ABARELIX® rmRH; lapatinib ditosylate (an ErbB-2 and EGFR dual tyrosine kinase small-molecule inhibitor also known as GW572016); and pharmaceutically acceptable salts, acids or derivatives of any of the above.

Chemotherapeutic agents also include antibodies such as alemtuzumab (Campath), bevacizumab (AVASTIN®); cetuximab (ERBITUX®); panitumumab (VECTIBIX®), rituximab (RITUXAN®), pertuzumab (OMNITARG®, 2C4), trastuzumab (HERCEPTIN®), tositumomab (Bexxar, Corixia), and the antibody drug conjugate, gemtuzumab ozogamicin (MYLOTARG®). Additional humanized monoclonal antibodies with therapeutic potential as agents in combination with the compounds of the invention include: apolizumab, aselizumab, atlizumab, bapineuzumab, bivatuzumab mertansine, cantuzumab mertansine, cedelizumab, certolizumab pegol, cidfusituzumab, cidtuzumab, daclizumab, eculizumab, efalizumab, epratuzumab, erlizumab, felvizumab, fontolizumab, gemtuzumab ozogamicin, inotuzumab ozogamicin, ipilimumab, labetuzumab, lintuzumab, matuzumab, mepolizumab, motavizumab, motovizumab, natalizumab, nimotuzumab, nolovizumab, numavizumab, ocrelizumab, omalizumab, palivizumab, pascolizumab, pecfusituzumab, pectuzumab, pexelizumab, ralivizumab, ranibizumab, reslivizumab, reslizumab, resyvizumab, rovelizumab, ruplizumab, sibrotuzumab, siplizumab, sontuzumab, tacatuzumab tetraxetan, tadocizumab, talizumab, tefibazumab, tocilizumab, toralizumab, tucotuzumab celmoleukin, tucusituzumab, umavizumab, urtoxazumab, ustekinumab, visilizumab, and the anti-interleukin-12 (ABT-874/J695) which is a recombinant exclusively human-sequence, full-length IgG1 A antibody genetically modified to recognize interleukin-12 p40 protein.

Chemotherapeutic agents also include “EGFR inhibitors,” which refers to compounds that bind to or otherwise interact directly with EGFR and prevent or reduce its signaling activity, and is alternatively referred to as an “EGFR antagonist.” Examples of such agents include antibodies and small molecules that bind to EGFR. Examples of antibodies which bind to EGFR include MAb 579 (ATCC CRL HB 8506), MAb 455 (ATCC CRL HB8507), MAb 225 (ATCC CRL 8508), MAb 528 (ATCC CRL 8509) (see, U.S. Pat. No. 4,943,533, Mendelsohn et al.) and variants thereof, such as chimerized 225 (C225 or Cetuximab; ERBUTIX®) and reshaped human 225 (H225) (see, WO 96/40210,); IMC-11F8, a fully human, EGFR-targeted antibody (Imclone); antibodies that bind type II mutant EGFR (U.S. Pat. No. 5,212,290); humanized and chimeric antibodies that bind EGFR as described in U.S. Pat. No. 5,891,996; and human antibodies that bind EGFR, such as ABX-EGF or Panitumumab (see WO98/50433, Abgenix/Amgen); EMD 55900 (Stragliotto et al. Eur. J. Cancer 32A:636-640 (1996)); EMD7200 (matuzumab) a humanized EGFR antibody directed against EGFR that competes with both EGF and TGF-alpha for EGFR binding; human EGFR antibody, HuMax-EGFR (GenMab); fully human antibodies known as E1.1, E2.4, E2.5, E6.2, E6.4, E2.11, E6.3, and E7.6. 3 and described in U.S. Pat. No. 6,235,883; MDX-447 (Medarex Inc); and mAb 806 or humanized mAb 806 (Johns et al., J. Biol. Chem. 279(29):30375-30384 (2004)). The anti-EGFR antibody may be conjugated with a cytotoxic agent, thus generating an immunoconjugate (see, e.g., EP 659,439A2, Merck Patent GmbH). EGFR antagonists include small molecules such as compounds described in U.S. Pat. Nos. 5,616,582, 5,457,105, 5,475,001, 5,654,307, 5,679,683, 6,084,095, 6,265,410, 6,455,534, 6,521,620, 6,596,726, 6,713,484, 5,770,599, 6,140,332, 5,866,572, 6,399,602, 6,344,459, 6,602,863, 6,391,874, 6,344,455, 5,760,041, 6,002,008, and 5,747,498, as well as the following PCT publications: WO 98/14451, WO 98/50038, WO 99/09016, and WO 99/24037. Particular small molecule EGFR antagonists include OSI-774 (CP-358774, erlotinib, TARCEVA®); PD 183805 (CI 1033, 2-propenamide, N-[4-[(3-chloro-4-fluorophenyl)amino]-7-[3-(4-morpholinyl)propoxy]-6-quin-azolinyl]-, dihydrochloride, Pfizer Inc.); ZD1839, gefitinib (IRESSA®) 4-(3′-Chloro-4′-fluoroanilino)-7-methoxy-6-(3-morpholinopropoxy)quinazolin,); ZM 105180 ((6-amino-4-(3-methylphenyl-amino)-quinazoline); BIBX-1382 (N8-(3-chloro-4-fluoro-phenyl)-N2-(1-methyl-piperidin-4-yl)-pyrimido[5,4-d]pyrimidine-2,8-diamine); PKI-166 ((R)-4-[4-[(1-phenylethyl)amino]-1H-pyrrolo[2,3-d]pyrimidin-6-yl]-phenol)-; (R)-6-(4-hydroxyphenyl)-4-[(1-phenylethyl)amino]-7H-pyrrolo[2,3-d]pyrimi-dine); CL-387785 (N-[4-[(3-bromophenyl)amino]-6-quinazolinyl]-2-butynamide); EKB-569 (N-[4-[(3-chloro-4-fluorophenyl)amino]-3-cyano-7-ethoxy-6-quinolinyl]-44-dimethylamino)-2-butenamide) (Wyeth); AG1478 (Pfizer); AG1571 (SU 5271; Pfizer); and dual EGFR/HER2 tyrosine kinase inhibitors such as lapatinib (TYKERB®, GSK572016 or N-[3-chloro-4-[(3 fluorophenyl)methoxy]phenyl]-6 [5 [[[2methyl sulfonyl)ethyl]amino]methyl]-2-furanyl]-4-quinazolinamine).

Chemotherapeutic agents also include “tyrosine kinase inhibitors” including the EGFR-targeted drugs noted in the preceding paragraph; small molecule HER2 tyrosine kinase inhibitors such as TAK165 available from Takeda; CP-724,714, an oral selective inhibitor of the ErbB2 receptor tyrosine kinase (Pfizer and OSI); dual-HER inhibitors such as EKB-569 (available from Wyeth) which preferentially binds EGFR but inhibits both HER2 and EGFR-overexpressing cells; lapatinib (GSK572016; available from Glaxo-SmithKline), an oral HER2 and EGFR tyrosine kinase inhibitor; PKI-166 (available from Novartis); pan-HER inhibitors such as canertinib (CI-1033; Pharmacia); Raf-1 inhibitors such as antisense agent ISIS-5132 available from ISIS Pharmaceuticals which inhibit Raf-1 signaling; non-HER targeted TK inhibitors such as imatinib mesylate (GLEEVEC®, available from Glaxo SmithKline); multi-targeted tyrosine kinase inhibitors such as sunitinib (SUTENT®, available from Pfizer); VEGF receptor tyrosine kinase inhibitors such as vatalanib (PTK787/ZK222584, available from Novartis/Schering AG); MAPK extracellular regulated kinase I inhibitor CI-1040 (available from Pharmacia); quinazolines, such as PD 153035,4-(3-chloroanilino) quinazoline; pyridopyrimidines; pyrimidopyrimidines; pyrrolopyrimidines, such as CGP 59326, CGP 60261 and CGP 62706; pyrazolopyrimidines, 4-(phenylamino)-7H-pyrrolo[2,3-d] pyrimidines; curcumin (diferuloyl methane, 4,5-bis (4-fluoroanilino)phthalimide); tyrphostines containing nitrothiophene moieties; PD-0183805 (Warner-Lamber); antisense molecules (e.g., those that bind to HER-encoding nucleic acid); quinoxalines (U.S. Pat. No. 5,804,396); tryphostins (U.S. Pat. No. 5,804,396); ZD6474 (Astra Zeneca); PTK-787 (Novartis/Schering AG); pan-HER inhibitors such as CI-1033 (Pfizer); Affinitac (ISIS 3521; Isis/Lilly); imatinib mesylate (GLEEVEC®); PKI 166 (Novartis); GW2016 (Glaxo SmithKline); CI-1033 (Pfizer); EKB-569 (Wyeth); Semaxinib (Pfizer); ZD6474 (AstraZeneca); PTK-787 (Novartis/Schering AG); INC-1C11 (Imclone), rapamycin (sirolimus, RAPAMUNE®); or as described in any of the following patent publications: U.S. Pat. No. 5,804,396; WO 1999/09016 (American Cyanamid); WO 1998/43960 (American Cyanamid); WO 1997/38983 (Warner Lambert); WO 1999/06378 (Warner Lambert); WO 1999/06396 (Warner Lambert); WO 1996/30347 (Pfizer, Inc); WO 1996/33978 (Zeneca); WO 1996/3397 (Zeneca) and WO 1996/33980 (Zeneca).

Chemotherapeutic agents also include dexamethasone, interferons, colchicine, metoprine, cyclosporine, amphotericin, metronidazole, alemtuzumab, alitretinoin, allopurinol, amifostine, arsenic trioxide, asparaginase, BCG live, bevacuzimab, bexarotene, cladribine, clofarabine, darbepoetin alfa, denileukin, dexrazoxane, epoetin alfa, elotinib, filgrastim, histrelin acetate, ibritumomab, interferon alfa-2a, interferon alfa-2b, lenalidomide, levamisole, mesna, methoxsalen, nandrolone, nelarabine, nofetumomab, oprelvekin, palifermin, pamidronate, pegademase, pegaspargase, pegfilgrastim, pemetrexed disodium, plicamycin, porfimer sodium, quinacrine, rasburicase, sargramostim, temozolomide, VM-26, 6-TG, toremifene, tretinoin, ATRA, valrubicin, zoledronate, and zoledronic acid, and pharmaceutically acceptable salts thereof.

Chemotherapeutic agents also include hydrocortisone, hydrocortisone acetate, cortisone acetate, tixocortol pivalate, triamcinolone acetonide, triamcinolone alcohol, mometasone, amcinonide, budesonide, desonide, fluocinonide, fluocinolone acetonide, betamethasone, betamethasone sodium phosphate, dexamethasone, dexamethasone sodium phosphate, fluocortolone, hydrocortisone-17-butyrate, hydrocortisone-17-valerate, aclometasone dipropionate, betamethasone valerate, betamethasone dipropionate, prednicarbate, clobetasone-17-butyrate, clobetasol-17-propionate, fluocortolone caproate, fluocortolone pivalate and fluprednidene acetate; immune selective anti-inflammatory peptides (ImSAIDs) such as phenylalanine-glutamine-glycine (FEG) and its D-isomeric form (feG) (IMULAN BioTherapeutics, LLC); anti-rheumatic drugs such as azathioprine, ciclosporin (cyclosporine A), D-penicillamine, gold salts, hydroxychloroquine, leflunomideminocycline, sulfasalazine, tumor necrosis factor alpha (TNF.alpha.) blockers such as etanercept (ENBREL®), infliximab (REMICADE®), adalimumab (HUMIRA®), certolizumab pegol (CIMZIA®), golimumab (SIMPONI®), Interleukin 1 (IL-1) blockers such as anakinra (KINERET®), T-cell co-stimulation blockers such as abatacept (ORENCIA®), Interleukin 6 (IL-6) blockers such as tocilizumab (ACTEMERA®); Interleukin 13 (IL-13) blockers such as lebrikizumab; Interferon alpha (IFN) blockers such as rontalizumab; beta 7 integrin blockers such as rhuMAb Beta7; IgE pathway blockers such as Anti-M1 prime; Secreted homotrimeric LTa3 and membrane bound heterotrimer LTa1/.beta.2 blockers such as Anti-lymphotoxin alpha (LTa); miscellaneous investigational agents such as thioplatin, PS-341, phenylbutyrate, ET-18-OCH3, and farnesyl transferase inhibitors (L-739749, L-744832); polyphenols such as quercetin, resveratrol, piceatannol, epigallocatechine gallate, theaflavins, flavanols, procyanidins, betulinic acid and derivatives thereof; autophagy inhibitors such as chloroquine; delta-9-tetrahydrocannabinol (dronabinol, MARINOL®); beta-lapachone; lapachol; cochicines; betulinic acid; acetylcamptothecin, scopolectin, and 9-aminocamptothecin); podophyllotoxin; tegafur (UFTORAL®); bexarotene (TARGRETIN®); bisphosphonates such as clodronate (for example, BONEFOS® or OSTAC®), etidronate (DIDROCAL®), NE-58095, zoledronic acid/zoledronate (ZOMETA®), alendronate (FOSAMAX®), pamidronate (AREDIA®), tiludronate (SKELID®), or risedronate (ACTONEL.theta.); and epidermal growth factor receptor (EGF-R); vaccines such as THERATOPE® vaccine; perifosine, COX-2 inhibitor (e.g., celecoxib or etoricoxib), proteosome inhibitor (e.g., PS341); CCI-779; tipifarnib (R11577); orafenib, ABT510; Bcl-2 inhibitor such as oblimersen sodium (GENASENSE®); pixantrone; farnesyltransferase inhibitors such as lonafarnib (SCH 6636, SARASAR™); and pharmaceutically acceptable salts, acids or derivatives of any of the above; as well as combinations of two or more of the above.

### EXAMPLES

### Example 1

### Iodide has Anti-Cachectic Activity

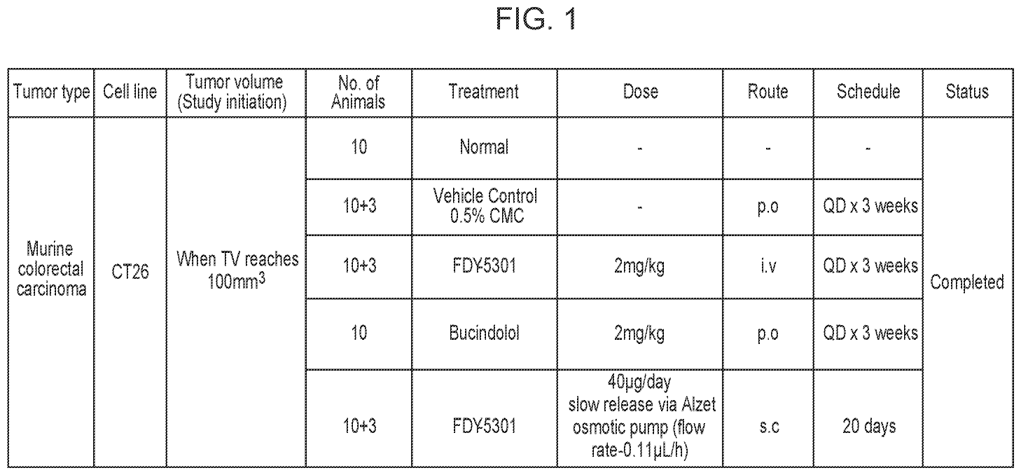

The anti-cachectic activity of iodide was evaluated in a cancer cachexia model of Balb/c mice bearing subcutaneous CT26 tumors. NaI was administered as FDY-5301, which is sodium iodide solubilized in water and balanced with sodium chloride to create an isotonic saline solution, with a pH between 7.0 and 9.5. As shown in the experimental design depicted in FIG. 1, when tumor volume reached 100 mm³, the mice were either left untreated, or treated with one of the following: (i) vehicle control (0.5% CMC); (ii) NaI (FDY-5301) at 2 mg/kg i.v., QD×3 weeks; (iii) bucindolol at 2 mg/kg p.o., QD×3 weeks; or (iv) NaI (FDY-5301) at 40 ug/day slow release via Alzet osmotic pump (flow rate 0.11 uL/h) s.c. for 20 days. On day 14, 1 hour post dosing, blood samples were taken from three animals from groups 2, 3, and 5. On day 20, the animals were humanely euthanized and tumor and animal characteristics were measured. Prior to euthanasia, blood samples were collected for evaluation of biochemical parameters. Following euthanasia, tumor tissue and organs, muscles were collected and weighed.

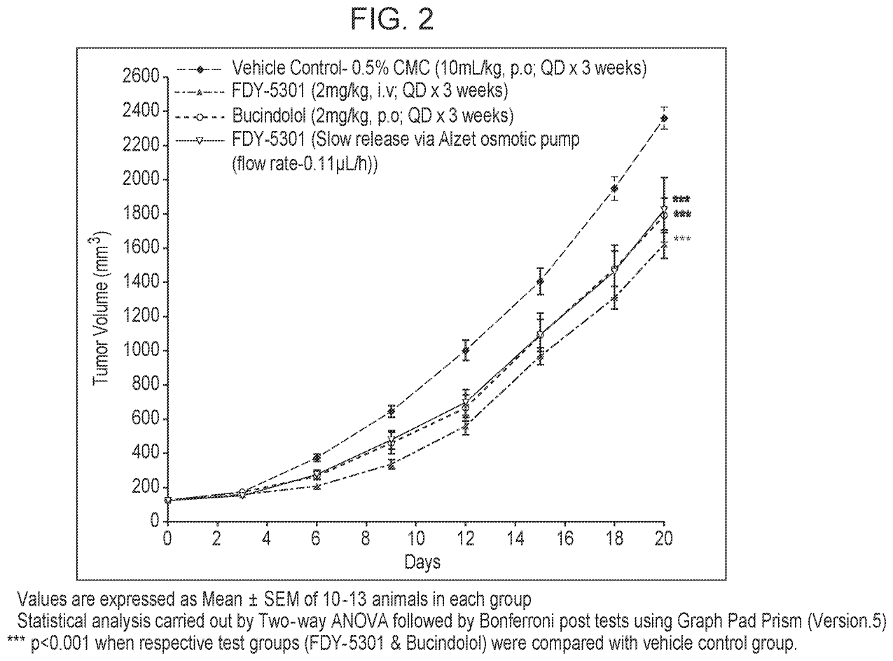

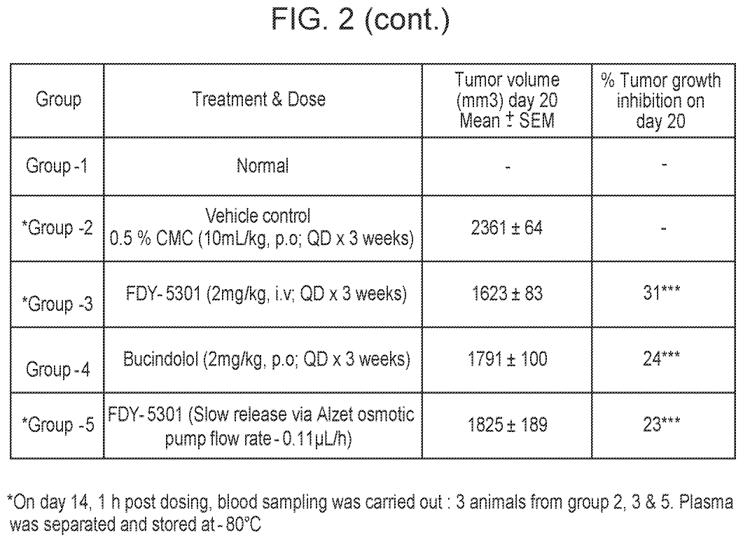

As shown in FIG. 2, treatment of groups 3, 4, and 5 resulted in significant inhibition of tumor growth. The difference in tumor volume was statistically significant when respective test groups (FDY-5301 and bucindolol) were compared with vehicle control group. All the tumor bearing groups had a significant increase in tumor volume over time, but no significant difference was observed among the treatment groups. Antitumor activity was evaluated as maximum tumor growth inhibition (TGI) in comparison to the vehicle control group. The % tumor grow inhibition (TGI) on day 20 for FDY-5301 (2 mg/kg, iv), bucindolol (2 mg/kg, po), and FDY-5301 (slow release via Alzet osmotic pump) treatment groups were found to be 31%, 24%, and 23%, respectively. The difference in tumor weight was statistically significant when respective test group were compared to vehicle control group.

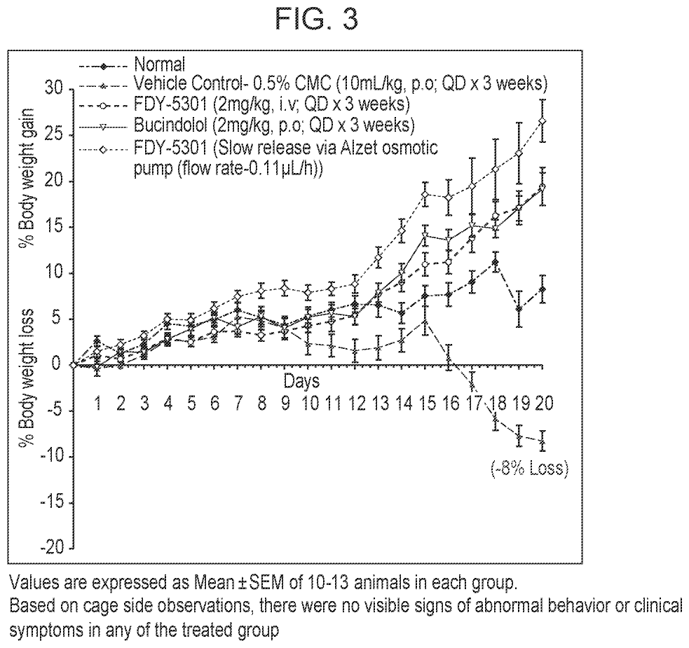

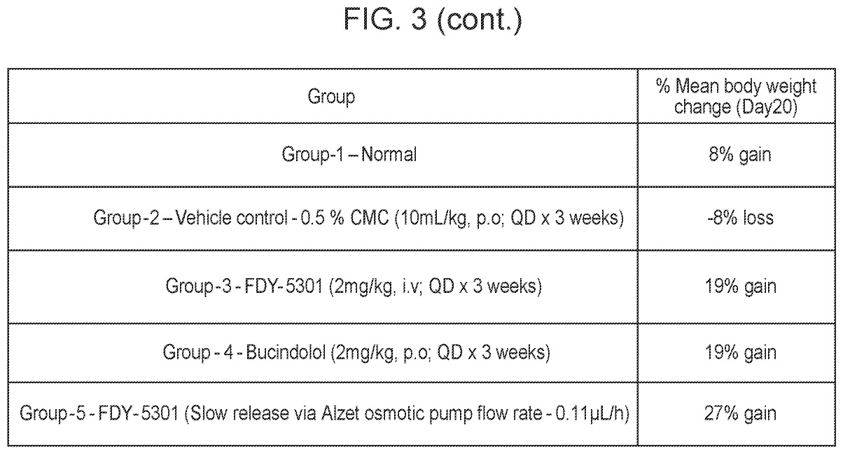

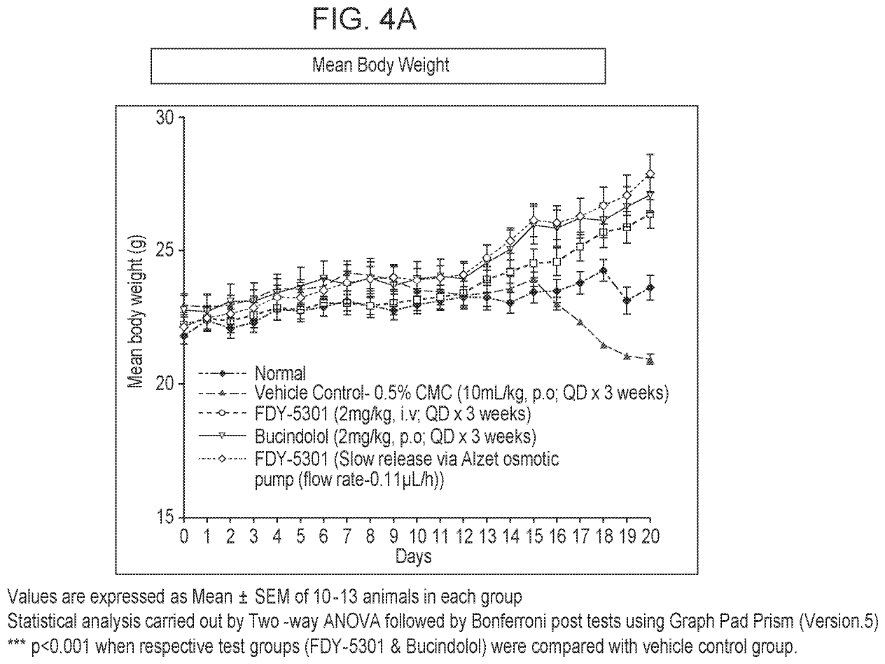

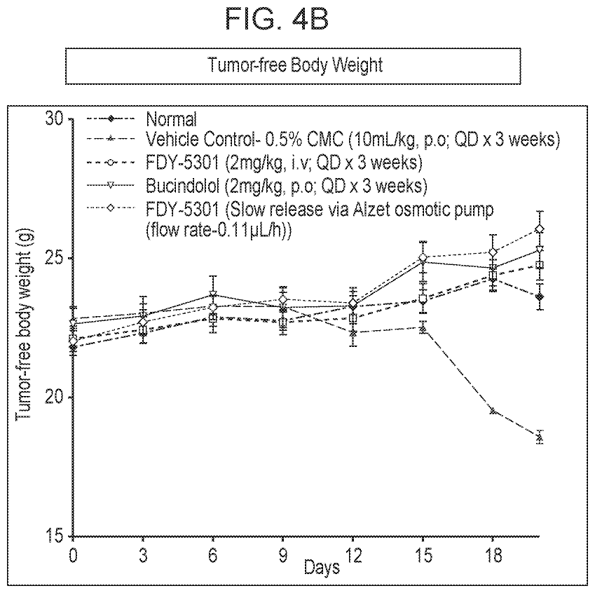

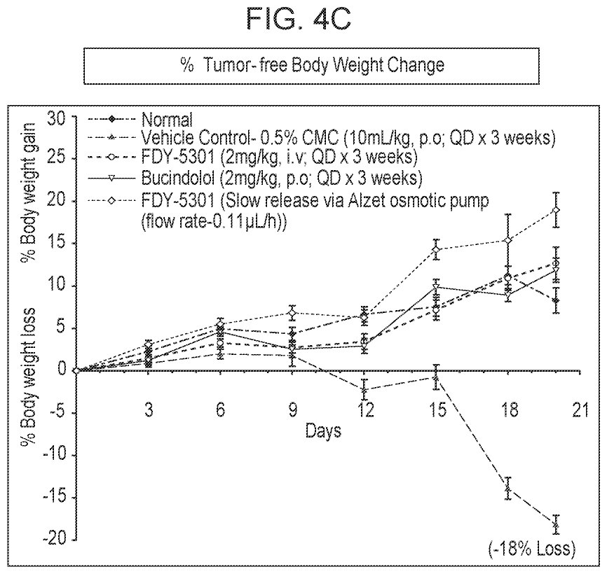

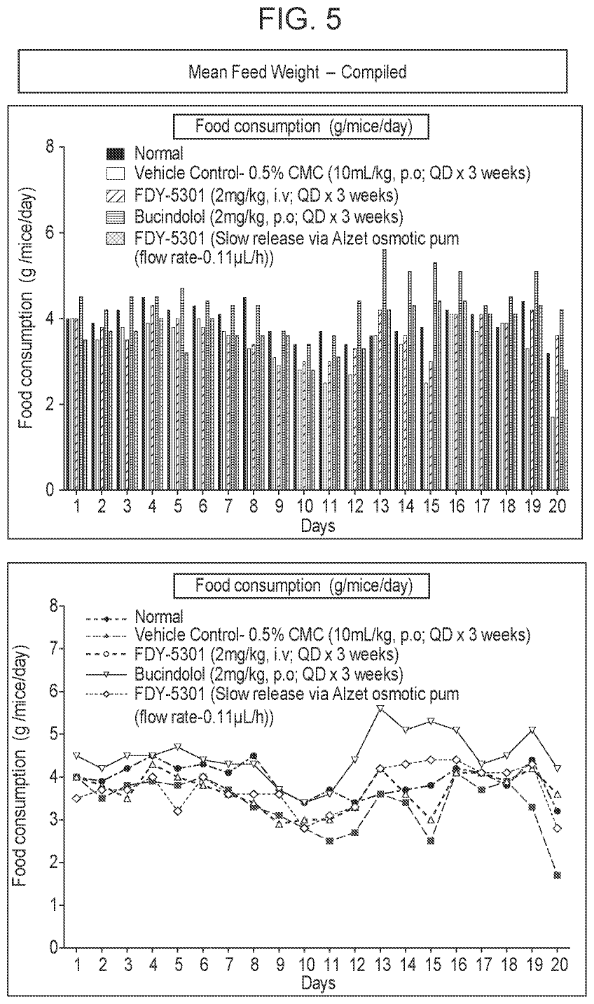

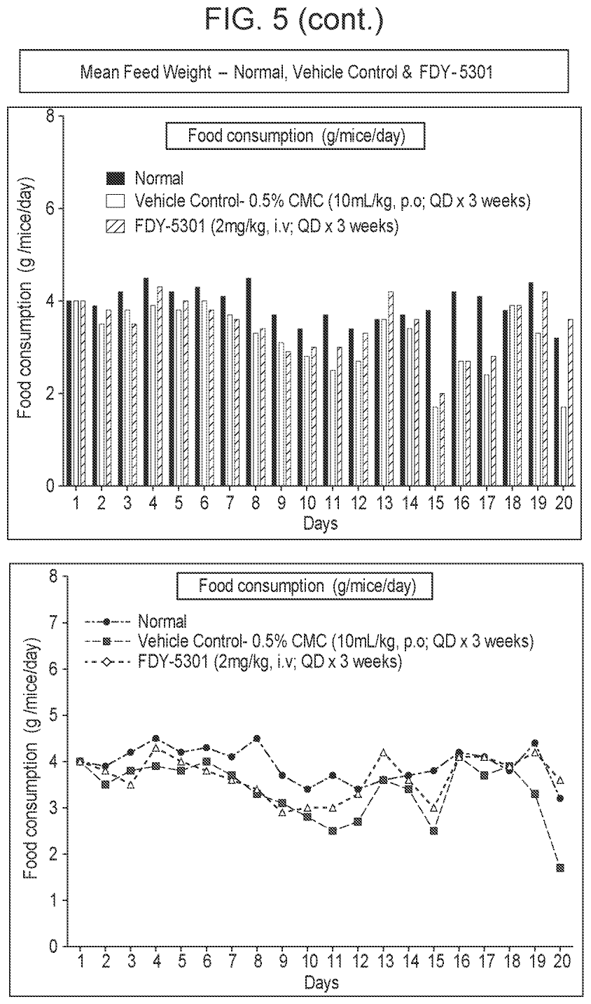

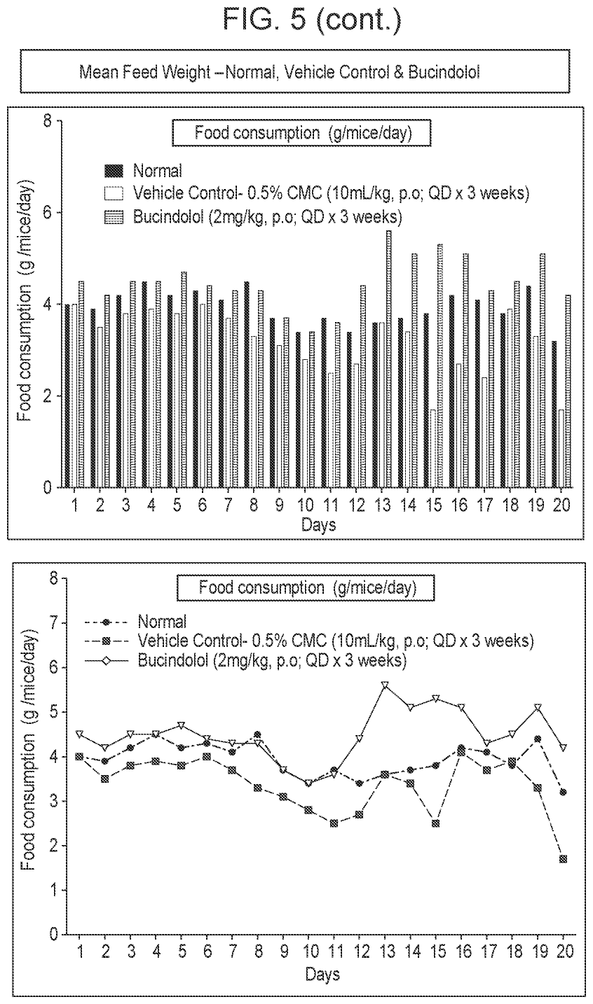

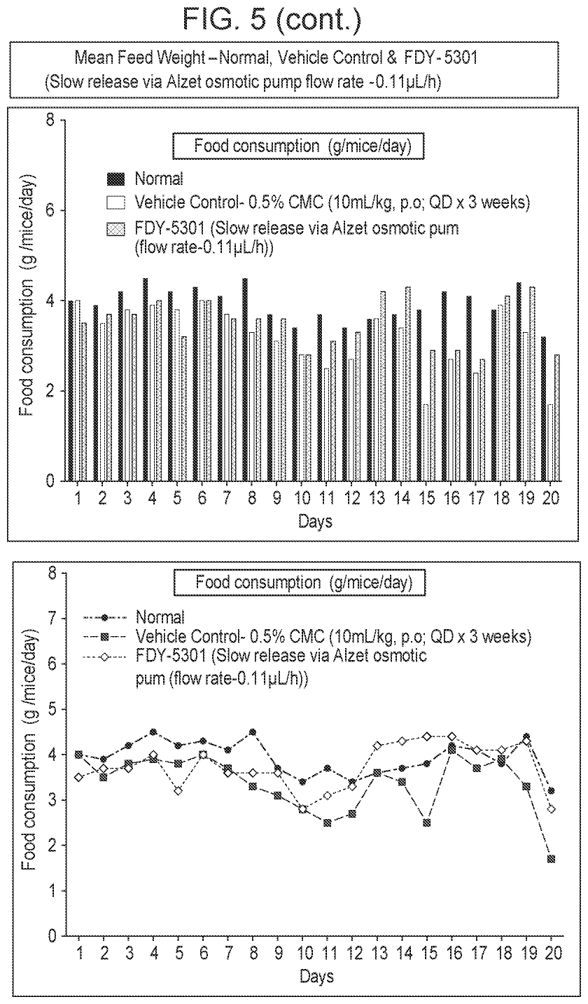

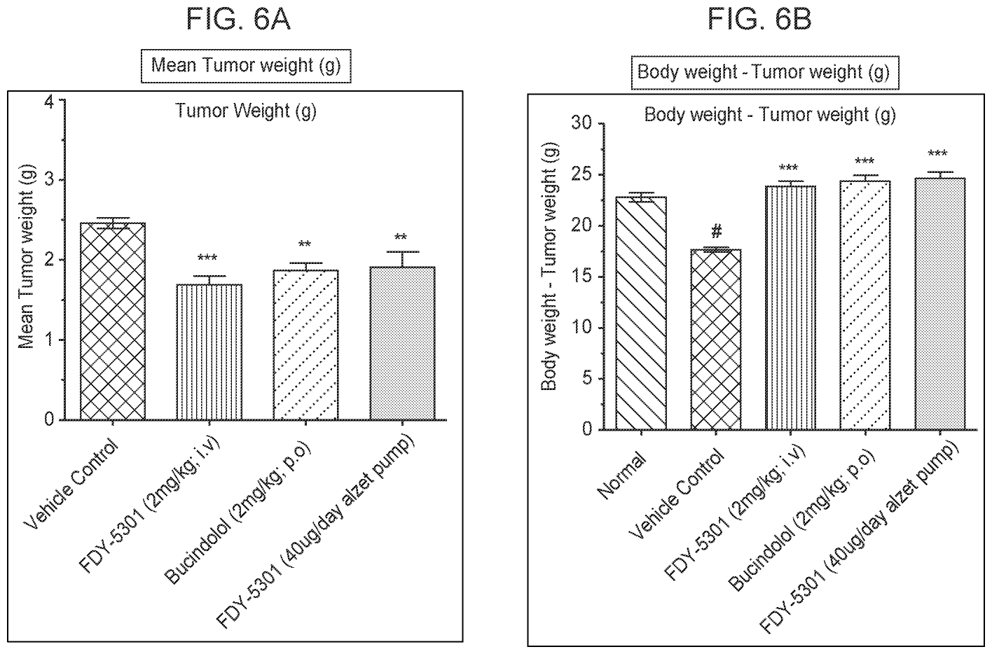

There was no difference in initial body weight among all groups. As the body weight of the normal group grew during the study period, the body weight of the vehicle control group increased during the initial nine days and then mild to moderate body weight loss was observed from day 10 until the end of treatment. There was significant % body weight loss observed in vehicle control group (−8% loss) when compared with the normal group. However, the treatment groups (FDY-5301 (2 mg/kg, iv), bucindolol (2 mg/kg, po), and FDY-5301 (slow release via Alzet osmotic pump)) showed progressive body weight gain throughout the experimental period. From day 12 to day 20, a significant decrease was observed in body weight of vehicle control animals (considering without tumor weight) compared to normal control animals. However, a significant increase in body weight (without tumor weight) was observed in all treatment groups as compared to vehicle control body weight. Of note, treatment of groups 3, 4 and 5 also resulted in increased body weight at day 20 (FIG. 3 and FIG. 4A). In addition, tumor-free body weight (FIG. 4B) and % tumor-free body weight change (FIG. 4C) both also increased for group 3, 4, and 5 treated animals, with the greatest change seen in group 5 animals (FIGS. 4A-4C). Food consumption during the experiment is shown in FIG. 5. While tumor weight was reduced in animals of treatment groups 3, 4, and 5 (FIG. 6A), body weight minus tumor weight was significantly greater in animals of treatment groups 3, 4, and 5, as compared to animals treated with vehicle control (group 2)(FIG. 6B).

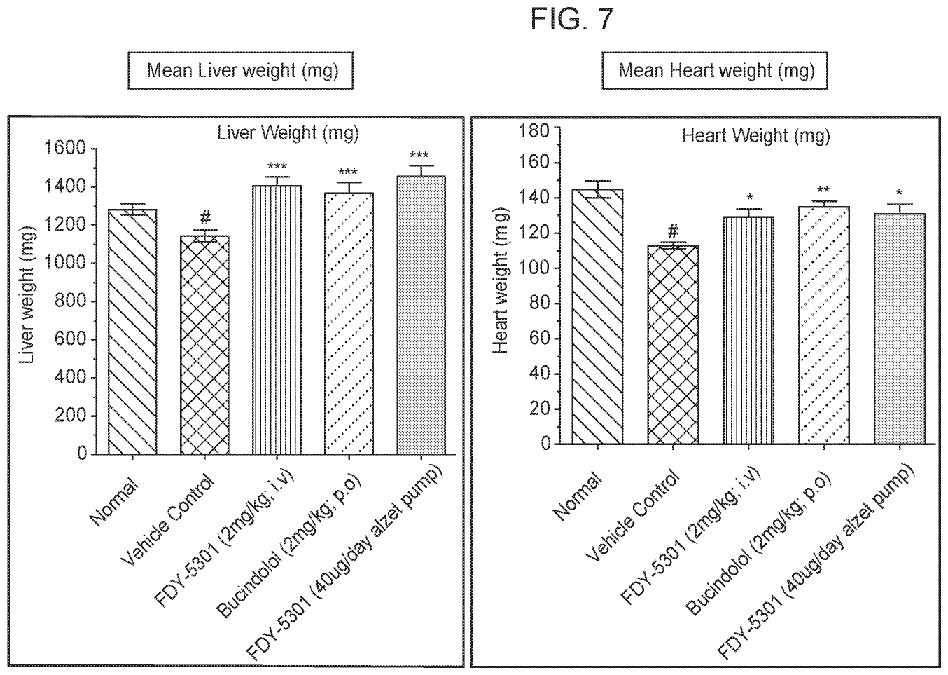

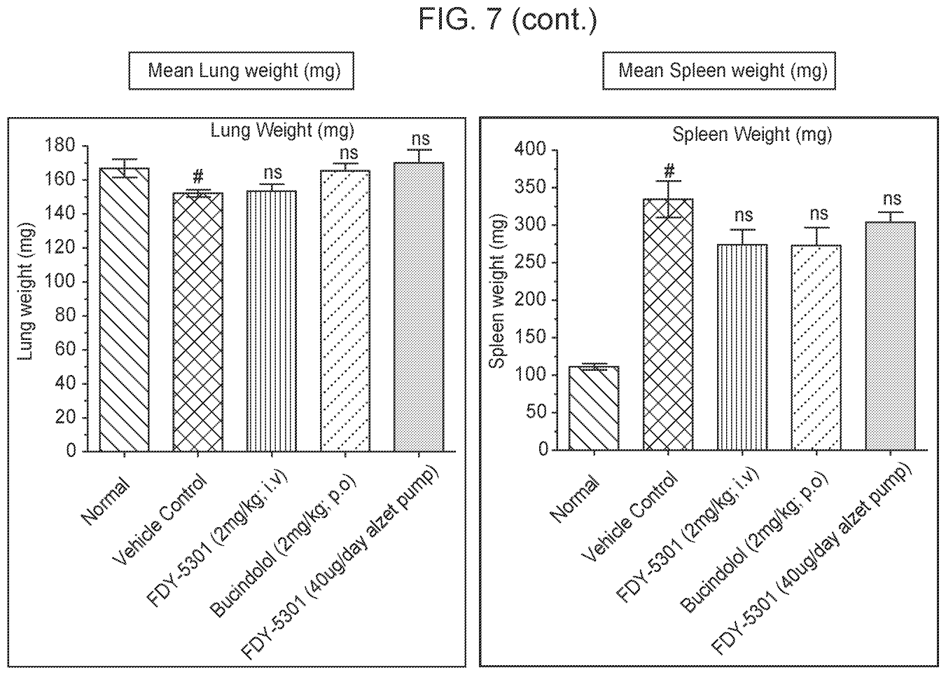

Examination of the weight of various organs demonstrated that animals of treatment groups 3, 4, and 5 had increased liver and heart weight as compared to animals treated with vehicle control (FIGS. 7A and 7B). Vehicle control (cachexia) group had no effect on the weight of the kidney. However, the weight of the liver, heart, lung and epididymal fat were significantly lower than the normal control group. The spleen weight was almost double in all the tumor bearing groups, which could be due to immune and inflammatory responses.

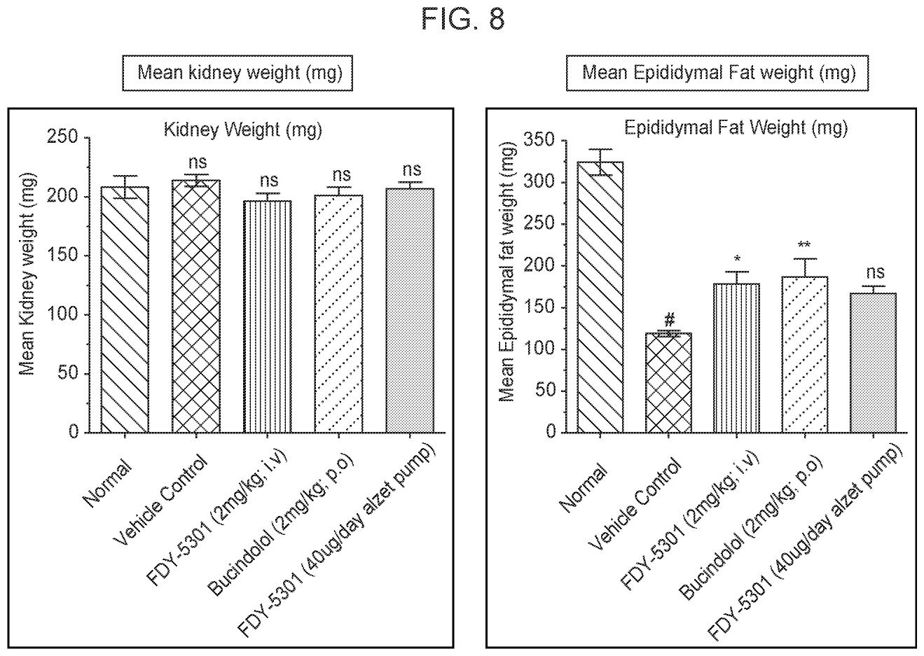

Similar examination of the weight of various muscles demonstrated that animals of treatment groups 3, 4, and 5 had increased tibialis anterior muscle weight as compared to animals treated with vehicle control (FIG. 8B). Vehicle control (cachexia) group showed significant weight loss in gastrocnemius, tibialis anterior and soleus muscles compared to the normal group. However, significant weight gain (moderate) in tibialis anterior muscle was observed in all treatment groups as compared to the vehicle control group. However, other muscles like gastrocnemius and soleus muscle weight was marginally higher in treatment groups as compared to the vehicle control group.

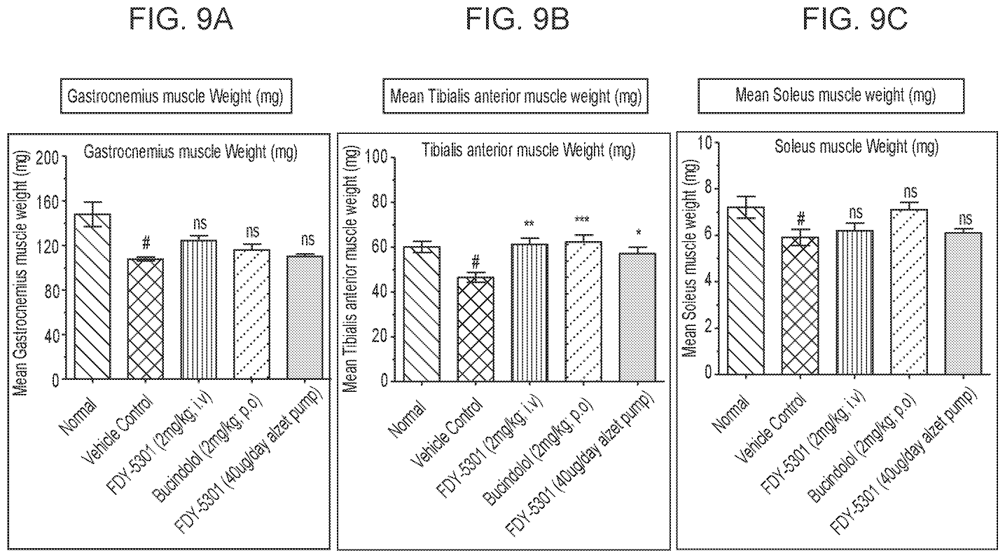

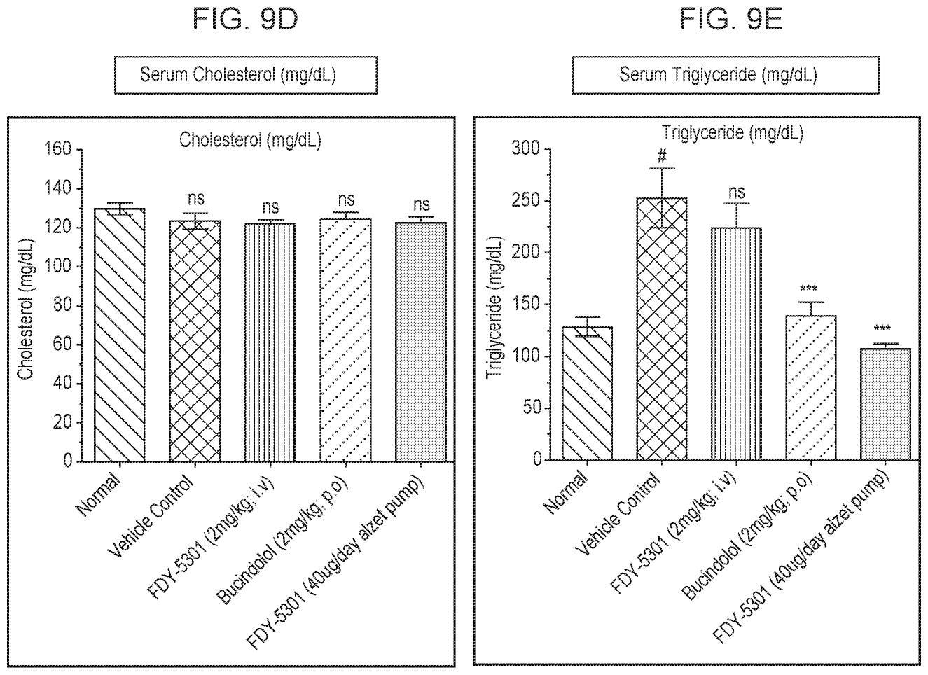

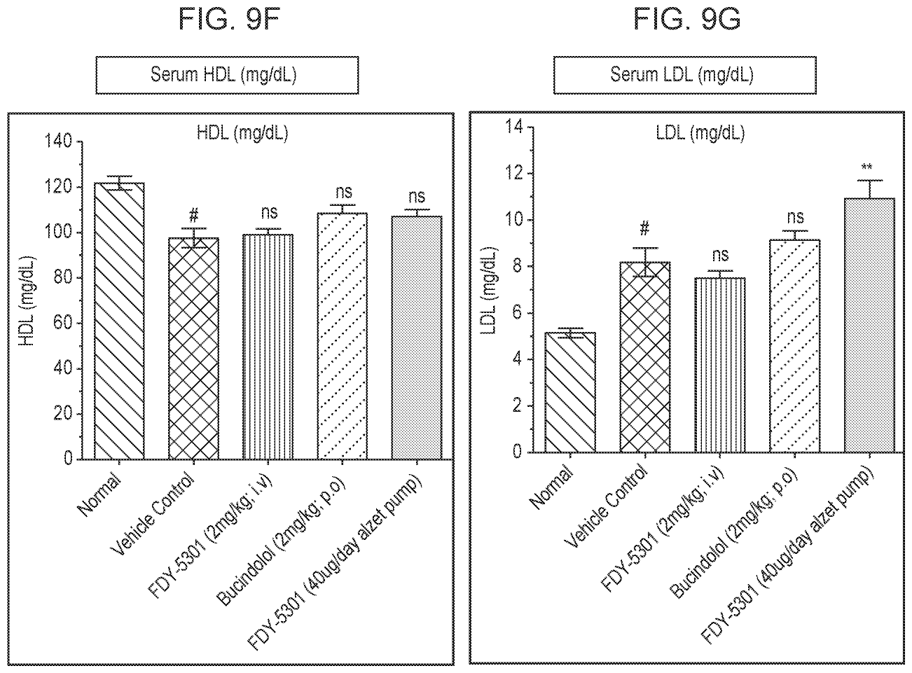

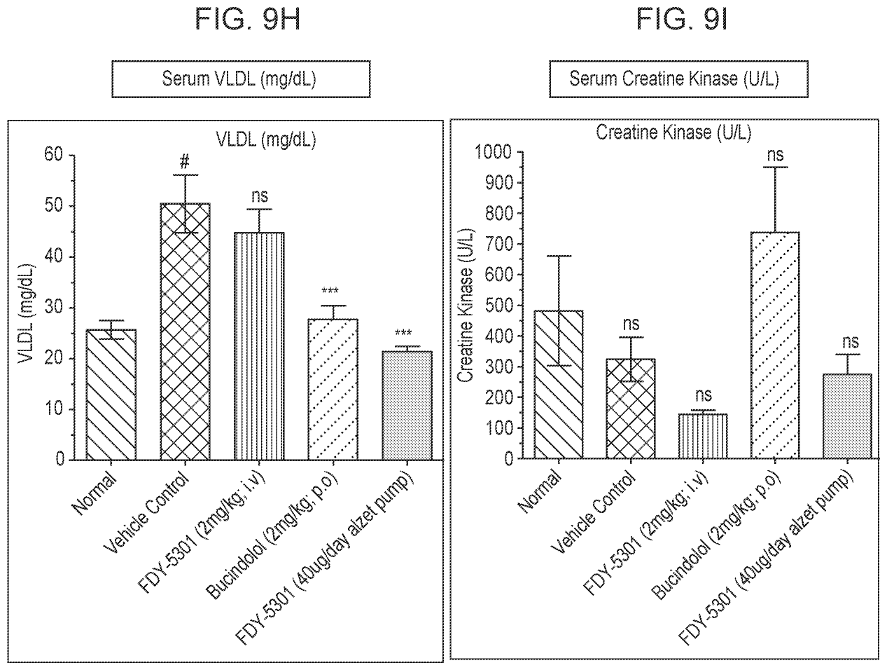

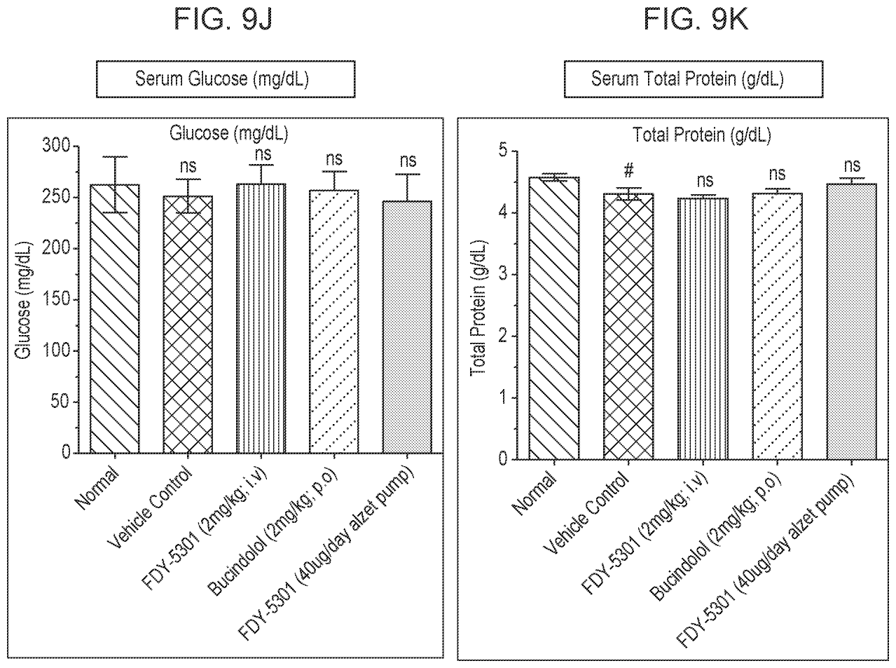

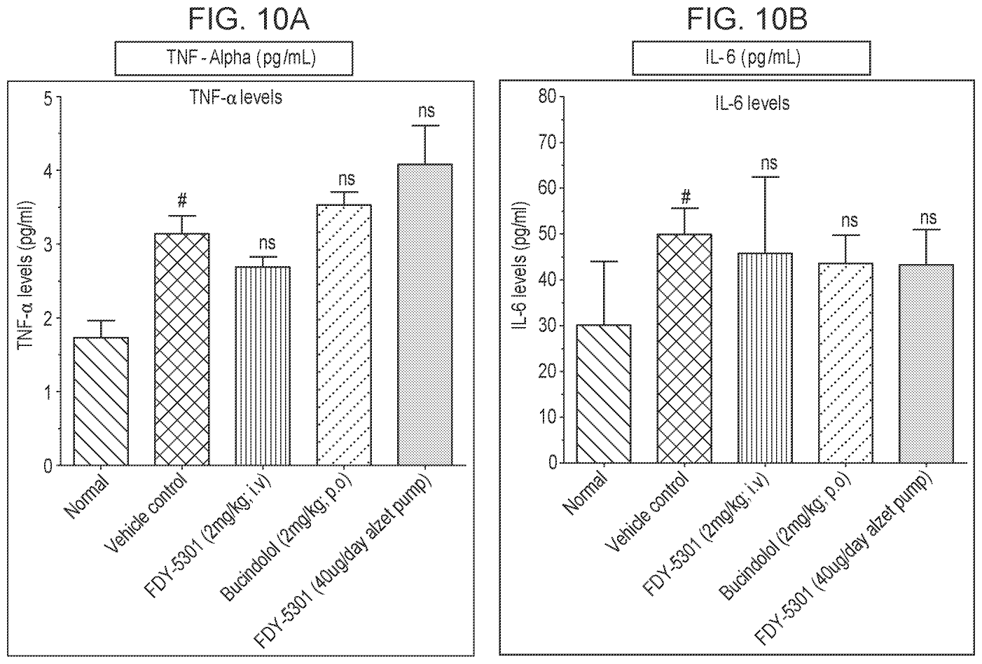

Biochemical analysis of the blood samples showed that while cholesterol, HDL, creatine kinase, glucose, and total protein levels were about the same in all animals, animals of treatment group 5 had lower triglyceride, higher LDL, and lower VLDL as compared to animals treated with vehicle control (FIGS. 9A-9H). Analysis of serum cytokine levels showed no significant difference in TNF-α or IL-6 levels between animals of treatment groups 3, 4, and 5 as compared to those treated with vehicle control (FIGS. 10A and 10B). There was no significant change in serum cholesterol or glucose levels in any of the groups. Serum triglyceride level in the vehicle control group was significantly high compared to the normal control group. However, triglyceride level in treatment with bucindolol and FDY-5301 (slow release via Alzet osmotic pump) was significantly lower and FDY-5301 (2 mg/kg, iv) marginally lower compared to the vehicle control group. Serum HDL level was significantly lower in the vehicle control group when compared to the normal control group. However, the serum HDL levels in all three treatment groups (3, 4, and 5) were marginally higher than the vehicle control group. Serum LDL level was significantly high in all treatment groups compared to normal control. Treatment with FDY-5301 (Slow release via Alzet osmotic pump) showed significant increase in LDL level compared to vehicle control. Whereas, non-significant change in LDL level was observed in other two treatment group (FDY-5301 (2 mg/kg, i.v), Bucindolol (2 mg/kg, p.o)) compared to vehicle control. Serum VLDL level was significantly high in vehicle control group compared to normal control. Whereas, the VLDL level was lower in all the treatment group. Bucindolol (2 mg/kg, p.o) & FDY-5301 (Slow release via Alzet osmotic pump) treatment showed significant decrease in VLDL level. Creatine kinase level in vehicle control group was non-significantly lower than normal control. Treatment with Bucindolol (2 mg/kg, p.o) showed higher level compared to vehicle control whereas, the treatment with FDY-5301 (2 mg/kg, i.v), & FDY-5301 (Slow release) showed marginal decrease levels were observed. The total protein level was significantly lower in vehicle control group compared to normal control, whereas, protein levels in all treatment groups were similar.

Plasma cytokine analysis showed a significant increase in TNF-α in vehicle control compared to normal control animals. The TNF-α level of FDY-5301 (2 mg/kg, iv) treatment group was non-significantly lower, whereas treatment with bucindolol (2 mg/kg, po) or FDY-5301 (slow release via Alzet osmotic pump) showed marginally higher levels of TNF-α when compared with the vehicle control group. IL-6 level was higher in all treatment groups, including vehicle control, when compared with normal control. However, IL-6 level was marginally lower in all treatment groups when compared to vehicle control.

### Example 2

### Muscle Histology and Morphometry

Morphometric evaluation of transverse sections of muscle fiber area (um2) was performed on tibialis anterior, gastrocnemius, and soleus muscles from the animals described in Example 1, for evaluation of anti-cachectic properties of FDY-5301 and bucindolol in the cancer cachexia model.

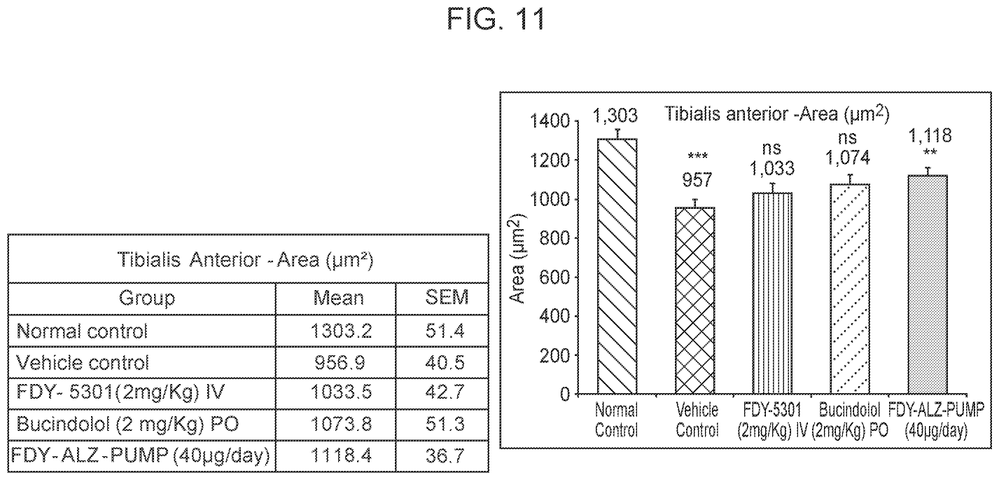

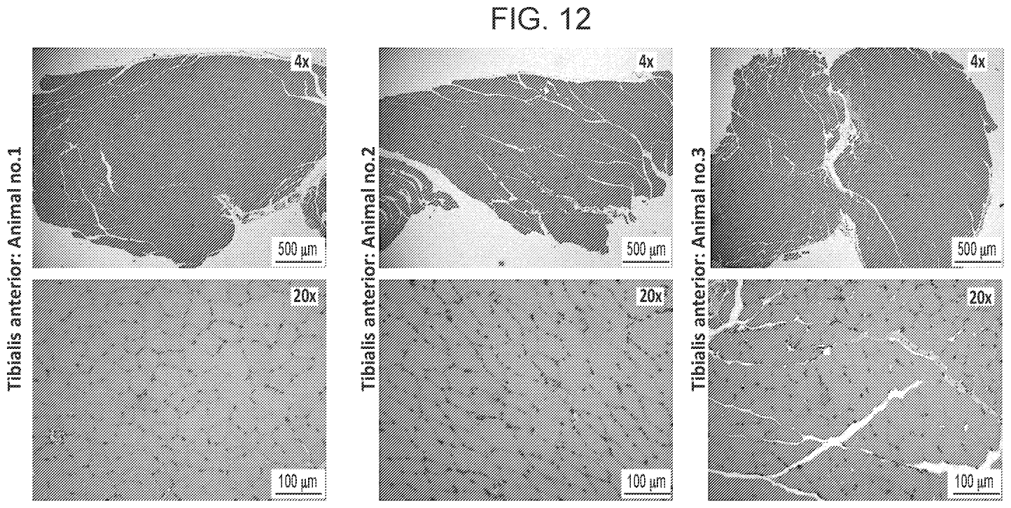

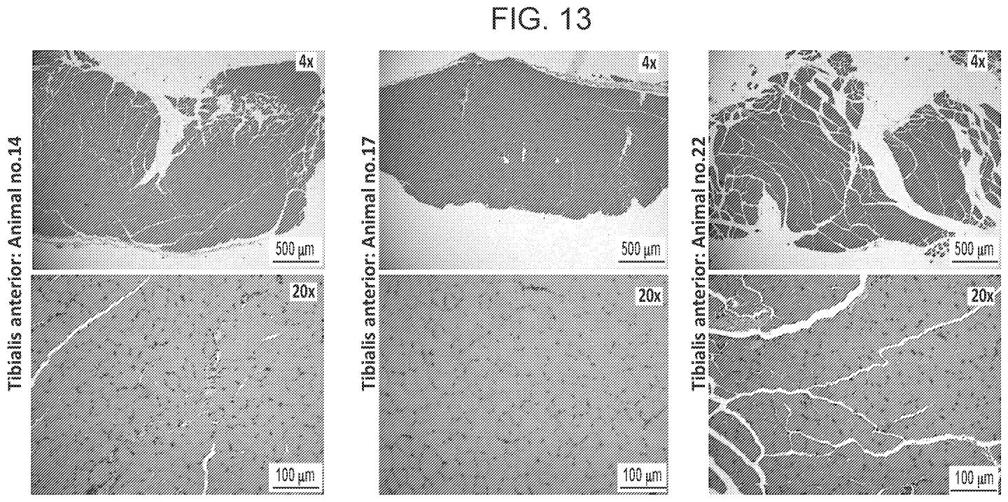

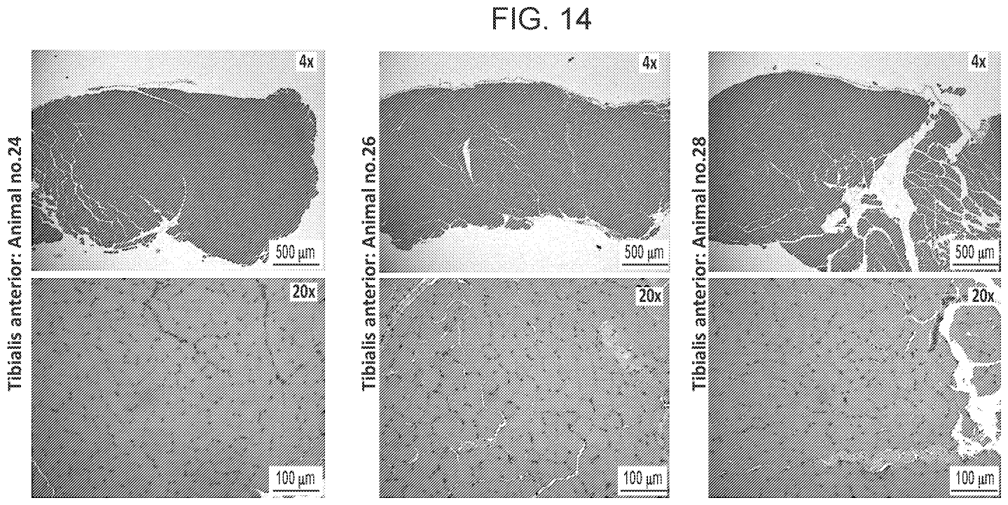

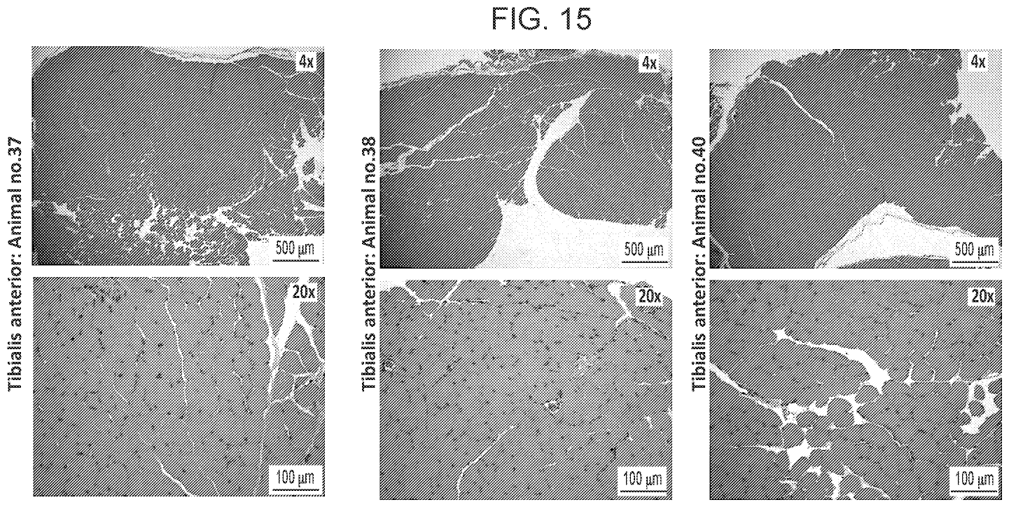

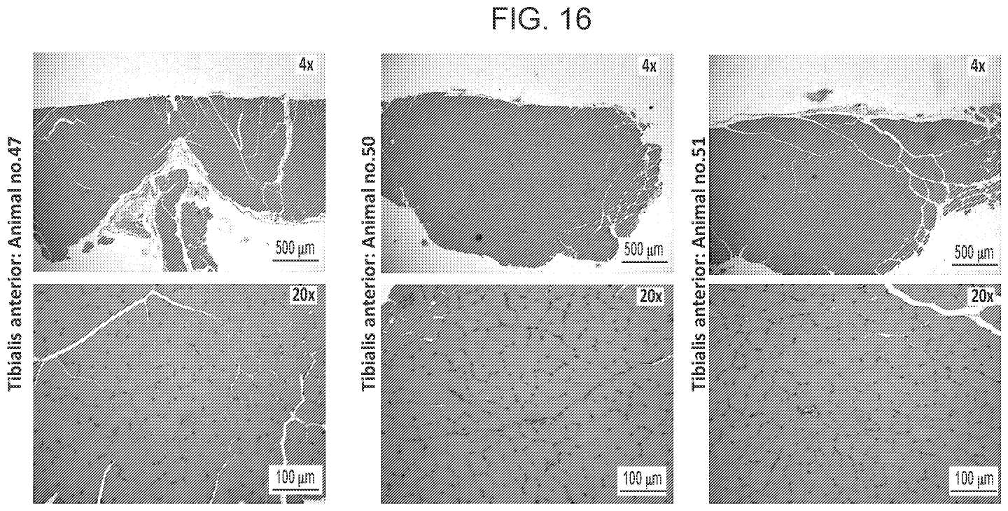

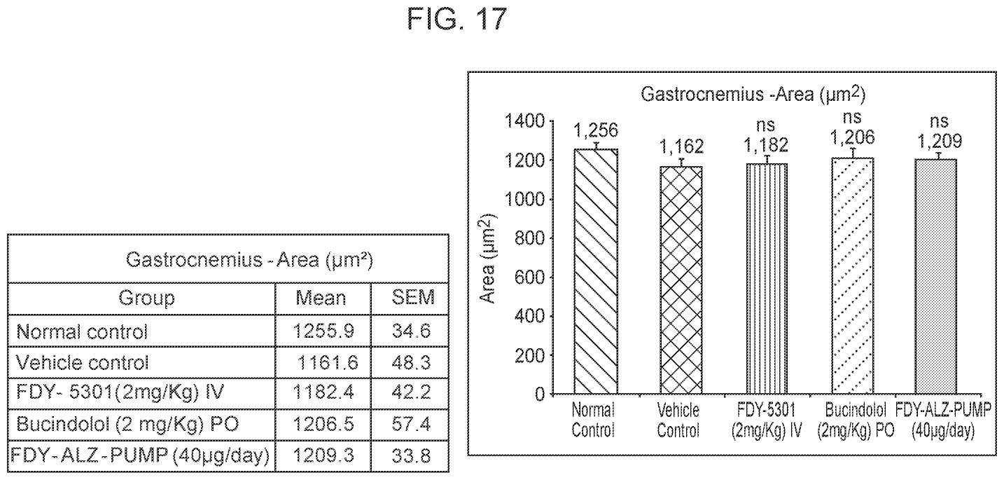

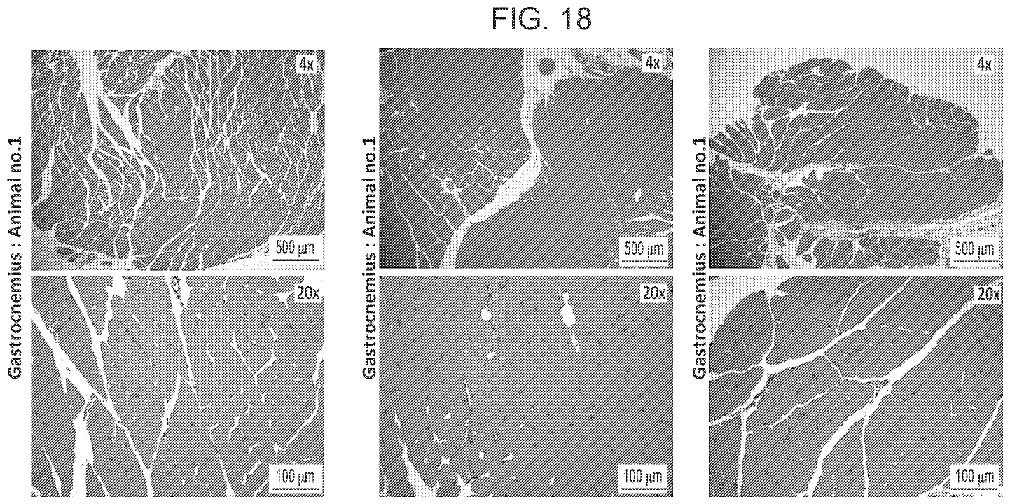

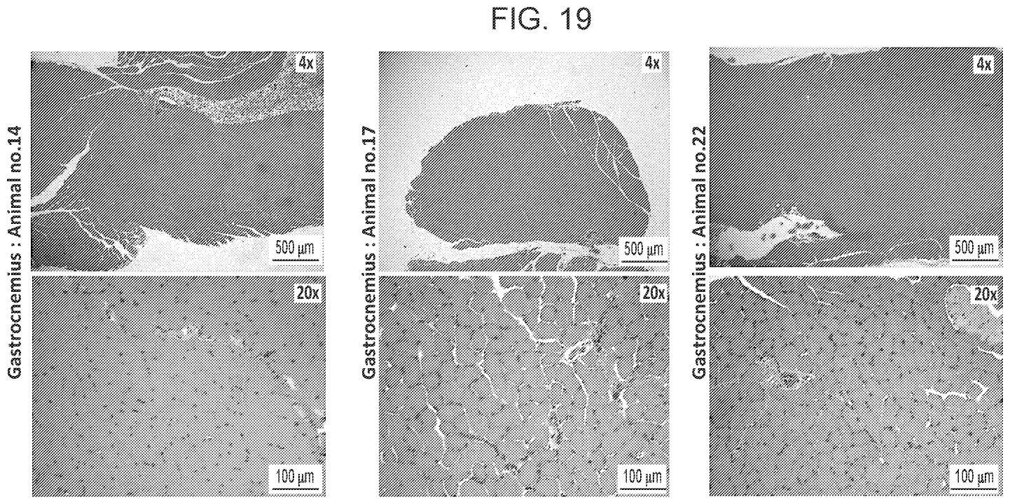

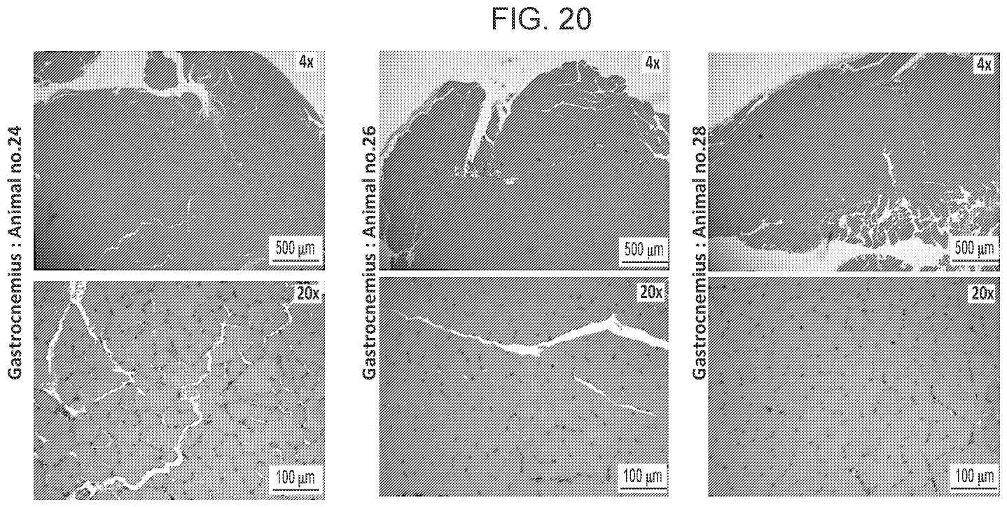

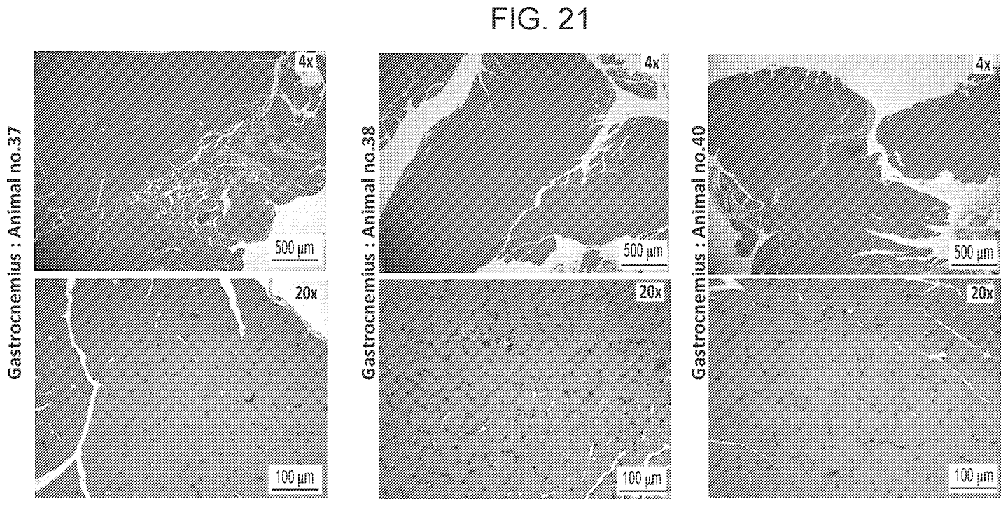

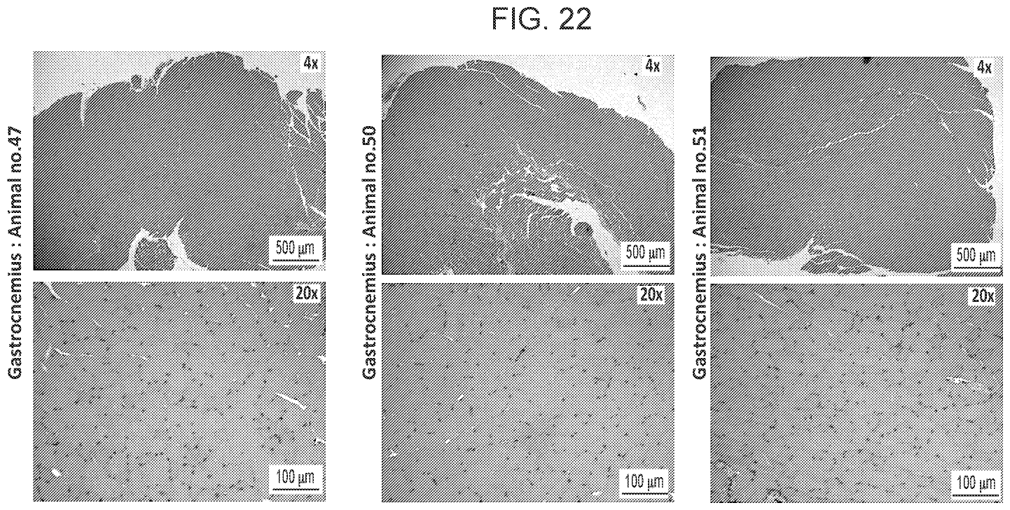

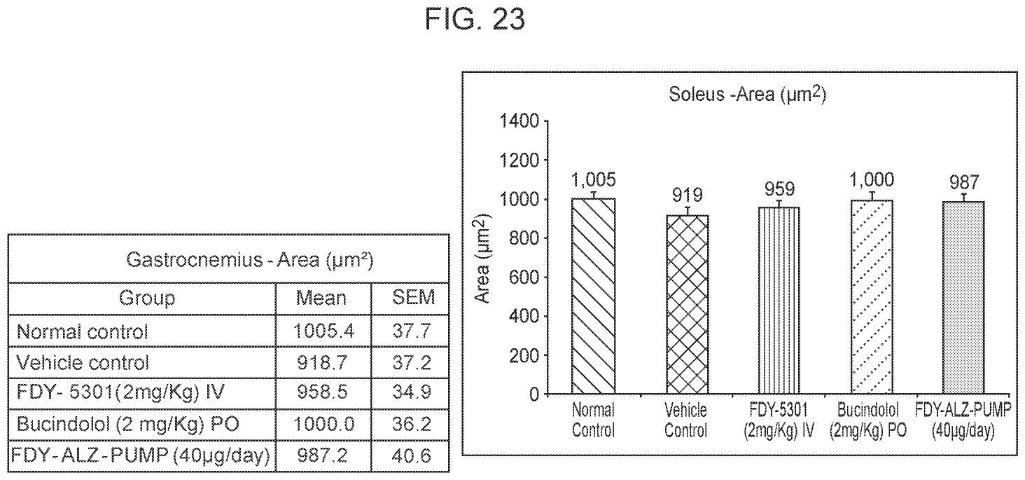

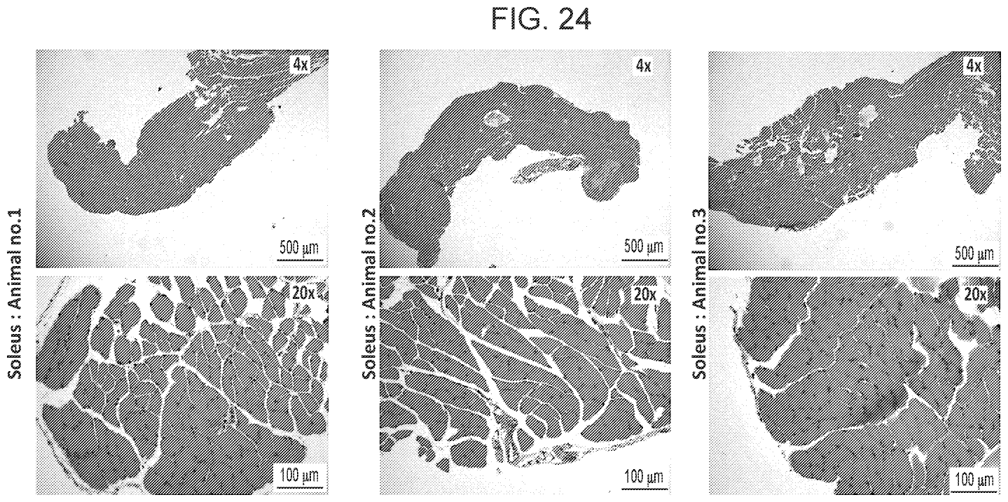

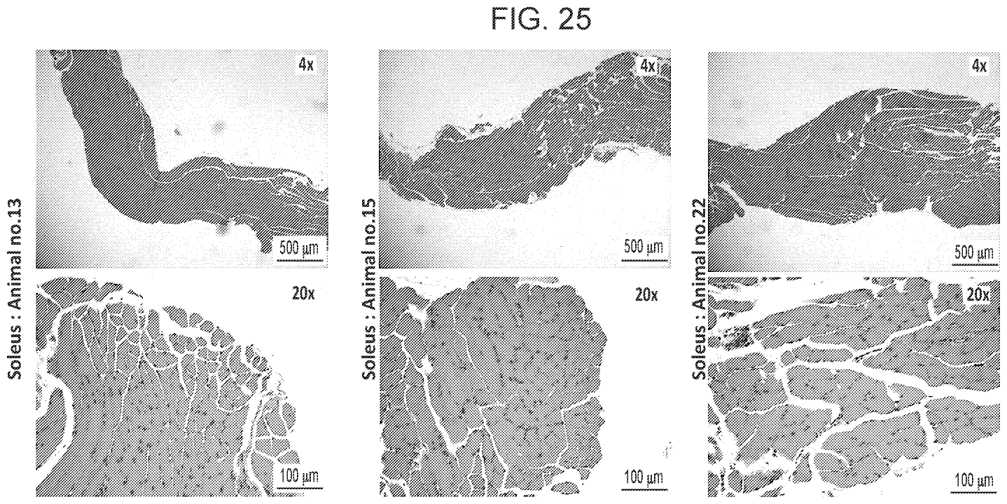

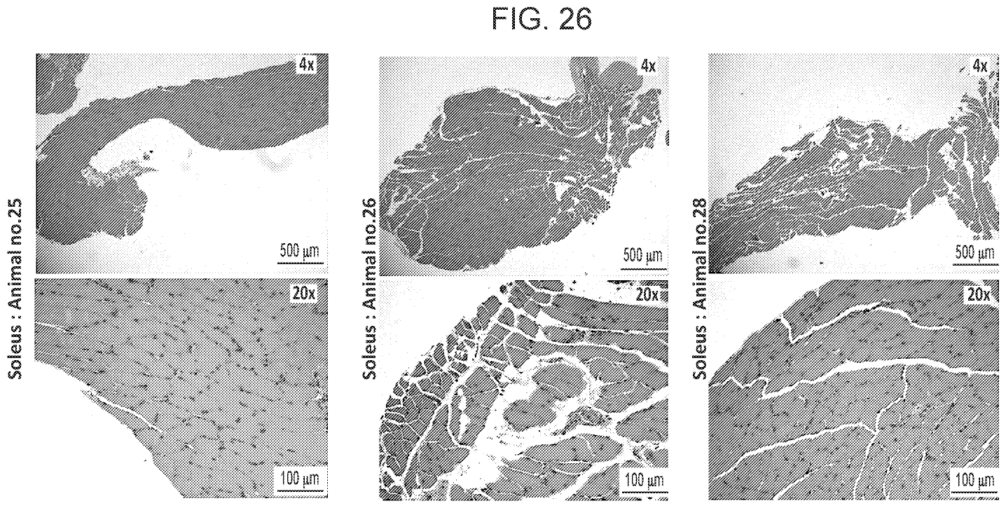

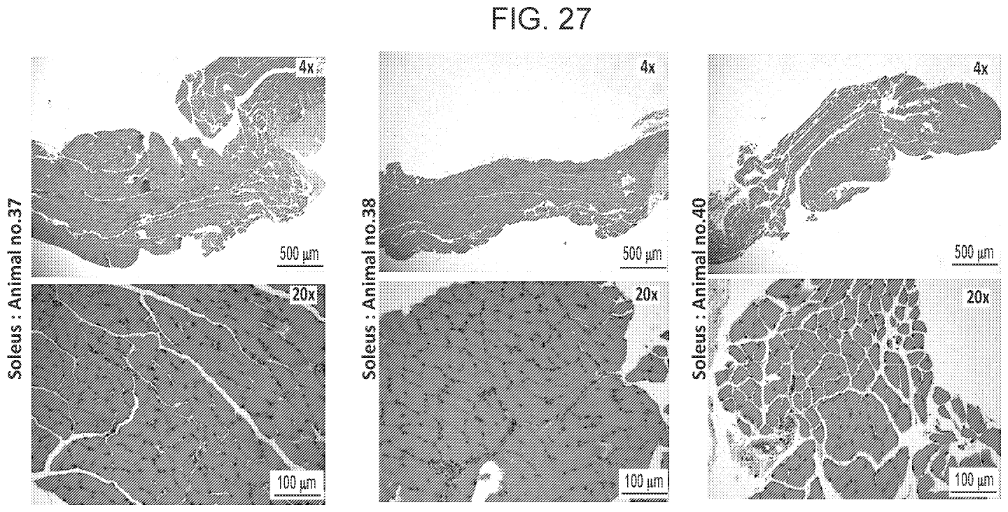

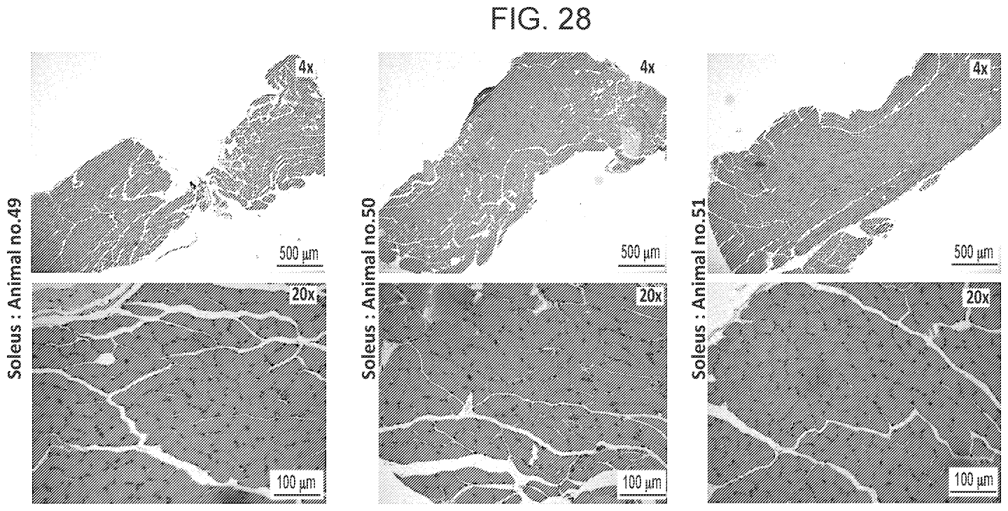

In all of the three muscles examined, there was a reduction in muscle fiber area in the vehicle control group as compared to the normal group (FIGS. 11, 17, and 23). In morphometric evaluation of tibialis anterior muscle, FDY-5301 (slow release via Alzet osmotic pump) showed significant increase in the muscle fiber area when compared to the vehicle control group (FIGS. 12-16). However, no significant increase in muscle fiber area was observed in gastrocnemius or soleus muscle when compared to the vehicle control group (FIGS. 18-22 and 24-28).

### Example 3

### Sodium Iodide Prevents Chemotherapy Induced Cardiotoxicity

This study demonstrates the efficacy of sodium iodide (FDY-5301) delivered by a single i.v. bolus or continuously (using a subcutaneous (s.c.) osmotic pump) in preventing cardiotoxicity induced by doxorubicin chemotherapy.

Male C57Bl/6 mice (˜10 weeks old) were given a 15 mg/kg i.p. bolus of doxorubicin to induce cardiotoxicity. Assessment of ejection fraction was performed via ultrasound at baseline (day 0, prior to any doxorubicin administration) and on days 3, 7, 14 or 28 days post administration. Dosing of placebo or FDY-5301 was done in a blinded fashion, as was all ultrasound analysis.

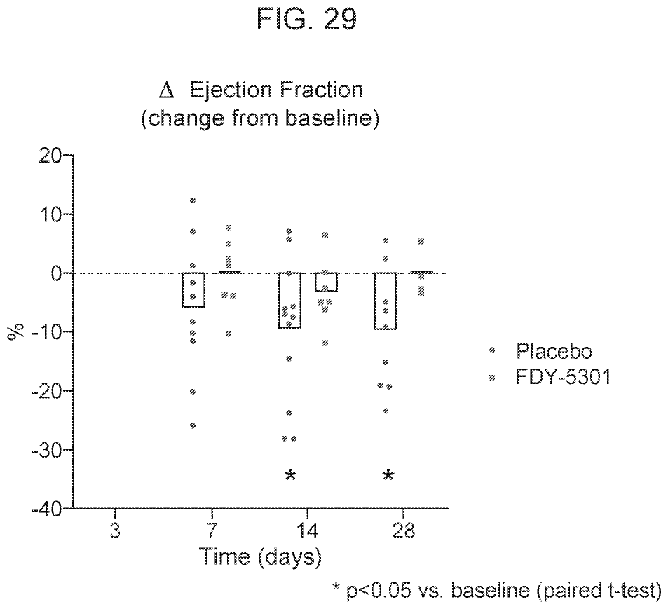

A single 2 mg/kg i.v. bolus of FDY-5301 was administered by the retro orbital (r.o.) route and immediately followed by 15 mg/kg doxorubicin i.p. Placebo treated animals received saline. A Vevo® 2100 imaging system was used to assess cardiac function on days 0 (baseline), 7, 14, & 28. As shown in FIG. 29, treatment with FDY-5301 was associated with less of a reduction in change in ejection fraction from baseline at days 7, 14, & 28.

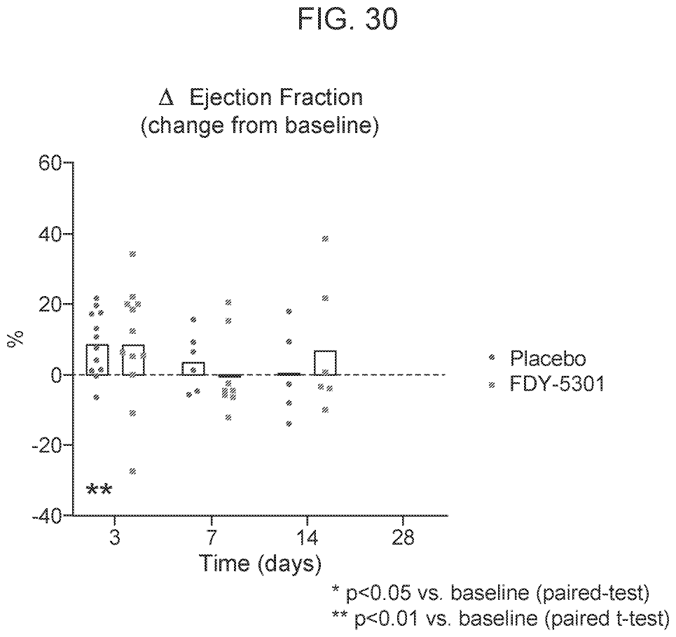

A single 2 mg/kg i.v. bolus of NaI was administered by the retro orbital (r.o.) route followed immediately by the s.c. implantation of an osmotic pump designed to deliver 2 mg/kg/day of NaI. The mice then received a 15 mg/kg doxorubicin i.p. bolus. Placebo treated animals received saline (and an osmotic pump filled with saline). Cardiac function was assessed on days 0 (baseline), 3, 7 & 14. As shown in FIG. 30, following a transient increase in EF (in both the placebo and FDY-5301 treated animals) treatment with FDY-5301 resulted in a stabilized EF (by day 14) while animals receiving placebo showed trends of a reduced EF on day 7 and further reductions on day 14.

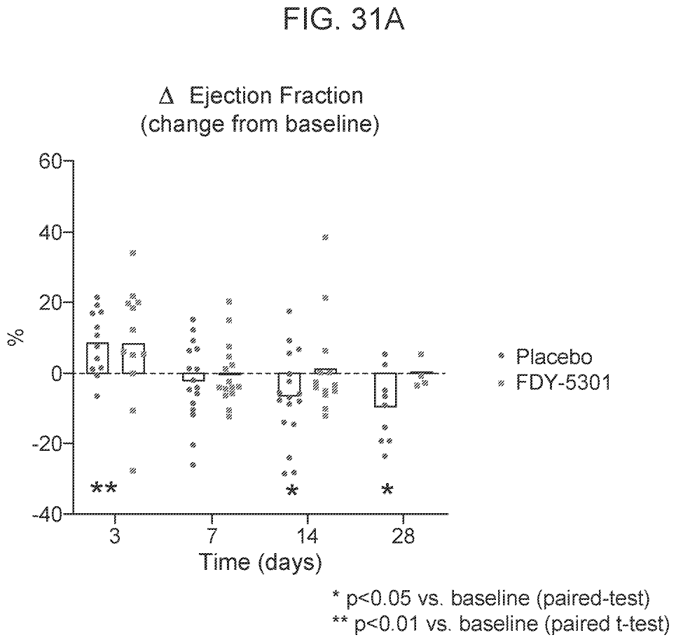

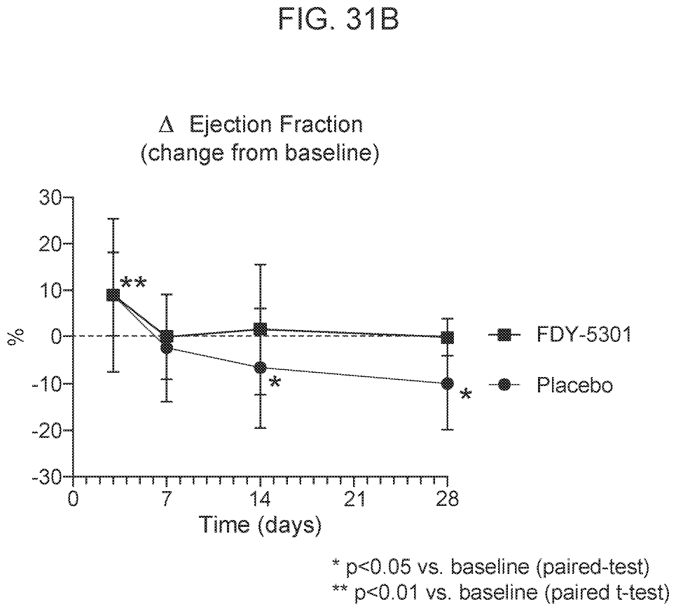

The combined results of these studies are shown in FIGS. 31A and 31B. Chemotherapy (doxorubicin) administration caused cardiotoxicity, e.g., reduced ejection fraction. This study shows that FDY-5301 delivered at the time of chemotherapy administration prevents a significant reduction in ejection fraction. Since protected ejection fraction is a measure of preserved (cardiac) muscle function, these results demonstrate that FDY-5301 is effective in preventing or reducing cardiotoxicity.

All of the above U.S. patents, U.S. patent application publications, U.S. patent applications, foreign patents, foreign patent applications and non-patent publications referred to in this specification and/or listed in the Application Data Sheet, are incorporated herein by reference, in their entirety.

From the foregoing it will be appreciated that, although specific embodiments of the invention have been described herein for purposes of illustration, various modifications may be made without deviating from the spirit and scope of the invention.

We claim:

1. A method for treating, inhibiting, reducing the severity of, or preventing cachexia or cardiotoxicity associated with or resulting from treatment of a subject with an anti-cancer therapy, comprising providing to the subject an effective amount of an iodide in combination with the anti-cancer therapy.

2. The method of claim 1, wherein the method is used for treating, inhibiting, reducing the severity of, or preventing cardiotoxicity.

3. The method of claim 1, wherein the method is for treating, inhibiting, reducing the severity of, or preventing cachexia, optionally cachexia of skeletal muscle or cachexia of cardiac muscle.

4. The method of any one of claims 1-3, wherein the subject is being treated for a cancer selected from the group consisting of: pancreatic cancer, bladder cancer, colorectal cancer, breast cancer, prostate cancer, renal cancer, hepatocellular cancer, lung cancer, ovarian cancer, cervical cancer, gastric cancer, esophageal cancer, head and neck cancer, melanoma, neuroendocrine cancer, central nervous system cancer, brain cancer, bone cancer, soft tissue sarcoma, non-small cell lung cancer, small-cell lung cancer, colon cancer, carcinoma, sarcoma, lymphoma, or leukemia.

5. The method of any one of claims 1-4, wherein the anti-cancer therapy comprises treatment with a chemotherapeutic agent.

6. The method of claim 5, wherein the chemotherapeutic agent is selected from the group consisting of: anthracyclines (optionally doxorubicin), cisplatin, cyclophosphamide, trastuzumab, paclitaxel, CPT-11, adriamycin, etoposide, 5-fluorouracil, and methotrexate.

7. The method of claim 6, wherein the chemotherapeutic agent is an anthracycline.

8. The method of claim 6, wherein the chemotherapeutic agents is doxorubicin.

9. The method of claim 6, wherein the chemotherapeutic agent is cisplatin.

10. The method of any one of claims 1-4, wherein the anti-cancer therapy comprises radiation therapy.

11. The method of any one of claims 1-10, wherein the iodide is sodium iodide.

12. The method of any one of claims 1-11, wherein the iodide, optionally sodium iodide, is provided to the subject in an amount sufficient to increase the blood concentration of the iodide in the subject by at least five-fold, at least ten-fold, at least 50-fold, at least 100-fold, at least 500-fold, at least 1000-fold, at least 10,000-fold, or at least 100,000-fold.

13. The method of any one of claims 1-12, wherein the iodide, optionally sodium iodide, and the anti-cancer agent are present in the subject during an overlapping time period.

14. The method of any one of claims 1-13, wherein the iodide, optionally sodium iodide, is provided to the subject before and/or during treatment of the subject with the anti-cancer agent.

15. The method of any one of claims 1-14, wherein the subject is provided with less than or equal to about 10 mg/kg of the iodide, optionally about 1.0 mg/kg or about 2.0 mg/kg of the iodide.

16. The method of claim 14 or claim 15, wherein the iodide, optionally sodium iodide, is provided to the subject at a dose of about 0.5 mg/kg to 5.0 mg/kg daily for a period of time during treatment of the subject with the anti-cancer agent.

17. The method of any one of claims 14-16, wherein the iodide, optionally sodium iodide, is provided to the subject as an intravenous bolus, optionally during a time period of about one hour to about one minute prior to treatment of the subject with the anticancer agent.

18. The method of any one of claims 1-17, wherein the iodide is present in a stable liquid pharmaceutical composition comprising the iodide compound and a pharmaceutically acceptable carrier, diluent, or excipient.

19. The method of claim 18, wherein at least 90% of the iodide in the composition is present in a reduced form for at least one hour, at least one week, at least one month, or at least six months when stored at room temperature.

20. The method of any one of claims 1-19, wherein said composition comprising the iodide comprises one or more of a reducing agent, a tonicity agent, a stabilizer, a surfactant, a lycoprotectant, a polyol, an antioxidant, or a preservative.

21. The method of any one of claims 1-20, wherein the iodide is provided to the subject orally or parenterally.

22. The method of any one of claims 16-21, wherein multiple doses of the iodide are provided to the subject.

23. The method of any one of claims 1-22, wherein the treatment with the iodide, optionally sodium iodide, results in a decreased loss or an increase in mean body weight as compared to in the absence of treatment with the iodide.

24. The method of any one of claims 1-23, wherein the treatment with the iodide, optionally sodium iodide, results in a decreased loss or an increase in tumor-free body weight as compared to in the absence of treatment with the iodide.

25. The method of any one of claims 1-23, wherein the treatment with the iodide, optionally sodium iodide, results in a decreased loss or an increase in liver weight, heart weight, and/or epididymal fat weight as compared to in the absence of treatment with the iodide.

26. The method of any one of claims 1-23, wherein the treatment with the iodide, optionally sodium iodide, results in a decreased loss or an increase in a muscle weight as compared to in the absence of treatment with the iodide.

27. The method of claim 26, wherein the muscle is tibialis anterior muscle.

28. The method of any one of claims 1-23, wherein the treatment with the iodide, optionally sodium iodide, results in decreased serum triglyceride levels, decreased serum VLDL levels, or increased serum LDL levels as compared to in the absence of treatment with the iodide.

29. The method of any one of claims 1-23, wherein the treatment with the iodide, optionally sodium iodide, results in a decreased tumor weight as compared to in the absence of treatment with the iodide.

30. The method of any one of claims 1-23, wherein the treatment with the iodide, optionally sodium iodide, results in a reduced or lessened ejection-fraction (e.g., LVEF) reduction, optionally an ejection-fraction (e.g., LVEF) reduction of less than 10% or less than 5%.

31. The method of any one of claims 1-23, wherein the treatment with the iodide, optionally sodium iodide, results in reduced cardiac dysfunction, optionally determined based on clinical symptoms or the use of echocardiogram or electrocardiogram (EKG).

32. The method of any one of claims 1-29, for treating cachexia, wherein the cachexia is precachexia with weight loss of less than 5%, cachexia with weight loss of 5% or greater, or refractory cachexia.

33. The method of any one of claim 1-23, 30 or 31, for treating cardiotoxicity, wherein the cardiotoxicity is reversible (type 2), irreversible (type 1), acute, chronic, or late-onset cardiotoxicity.
